(19) United States
(12) Patent Application Publication
(10) Pub. No.: US 2023/0390538 A1
(43) Pub. Date: Dec. 7, 2023

(54) CARDIAC SHUNT DEVICE TO MINIMIZE DISRUPTION AND ENHANCE NATURAL FLOW PATTERNS OF BLOOD THROUGH THE HEART

(71) Applicant: EDWARDS LIFESCIENCES CORPORATION, Irvine, CA (US)

(72) Inventors: John Charles Laschinger, Laguna Beach, CA (US); Anthony Ciro Vrba, San Clemente, CA (US); Mazen Saadi Albaghdadi, Irvine, CA (US)

(21) Appl. No.: 18/330,283

(22) Filed: Jun. 6, 2023

Related U.S. Application Data
(60) Provisional application No. 63/349,807, filed on Jun. 7, 2022.

(51) Int. Cl. A61M 27/00

(52) U.S. Cl. CPC A61M 27/002; A61M 2210/125

(57) ABSTRACT

A method of selecting a shunt device for implantation in a heart includes obtaining a first MRI of the heart, and generating a simulation of flow patterns of blood flow in the heart. Blood flow in the heart is simulated when various shunt devices are implanted in the heart. The shunt device that complements the flow patterns of blood flow in the heart is selected, and the shunt device is implanted in the heart.

### CROSS-REFERENCE TO RELATED APPLICATION(S)

This application claims priority to U.S. Provisional Application No. 63/349,807, filed on Jun. 7, 2022, and entitled “Cardiac Shunt Device to Minimize Disruption To and Enhance Natural Flow Patterns of Blood Through the Heart,” the disclosure of which is hereby incorporated by reference in its entirety.

### BACKGROUND

The present disclosure relates to cardiac shunt devices, and in particular, to a cardiac shunt device for reducing left atrial pressure.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt device can be positioned in the septal wall between the left atrium and the right atrium to shunt blood from the left atrium into the right atrium, thus reducing the pressure in the left atrium.

### SUMMARY

A method of selecting a shunt device for implantation in a heart includes obtaining a first MRI of the heart, and generating a simulation of flow patterns of blood flow in the heart. Blood flow in the heart is simulated when various shunt devices are implanted in the heart. The shunt device that complements the flow patterns of blood flow in the heart is selected, and the shunt device is implanted in the heart.

A method of shunting blood between a left atrium and a right atrium of a heart includes positioning a shunt device in a tissue wall between the left atrium and a coronary sinus of the heart so that a flow path through a central flow tube of the shunt device is positioned to guide a flow of blood through the flow path of the shunt device to join with a natural flow pattern of blood flow within the coronary sinus. The blood is shunted from the left atrium to the coronary sinus through the flow path of the shunt device. The flow of blood through the shunt device is joined with the natural flow pattern of blood flow within the coronary sinus. The blood from the coronary sinus is moved into the right atrium via a natural orifice of the coronary sinus. The flow of blood from the coronary sinus is joined with a natural flow pattern of blood flow within the right atrium.

A method of shunting blood between a left atrium and a right atrium of a heart includes positioning a shunt device in a tissue wall between the left atrium and a coronary sinus of the heart so that a flow path through a central flow tube of the shunt device guides a left-sided flow vortex of blood flow within the left atrium through the central flow tube of the shunt device and is positioned to guide the flow of blood through the flow path of the shunt device to join with a helical flow pattern of blood flow within the coronary sinus. The blood is shunted from the left atrium to the coronary sinus through the flow path of the shunt device. The flow of blood through the shunt device is joined with the helical flow pattern of blood flow within the coronary sinus. The blood from the coronary sinus is moved into the right atrium. The flow of blood from the coronary sinus is joined with a natural flow pattern of blood flow within the right atrium.

A method of shunting blood between a left atrium and a right atrium of a heart includes positioning a shunt device in a tissue wall between the left atrium and a coronary sinus of the heart so that a flow path through a central flow tube of the shunt device guides a natural flow pattern of blood flow with the left atrium through the central flow tube of the shunt device. The blood is shunted from the left atrium to the coronary sinus through the flow path of the shunt device. The flow of blood through the shunt device is joined with a natural flow pattern of blood flow within the coronary sinus. The blood is moved from the coronary sinus into the right atrium, and the flow of blood from the coronary sinus is joined with a natural flow pattern of blood flow within the right atrium.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood flow within a coronary sinus.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood flow within a left atrium.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube is angled between 150 and 900 with respect to the tissue wall.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. The flow path has a diameter between 0.12 inches (3 millimeters) and 0.39 inches (10 millimeters). When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood in a coronary sinus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a heart and vasculature.

FIG. 2 is a cross-sectional schematic view of the heart.

FIG. 3A is a first schematic diagram illustrating modeled hemodynamic flow patterns in a heart.

FIG. 3B is a second schematic diagram illustrating modeled hemodynamic flow patterns in a heart.

FIG. 4A is a first schematic diagram illustrating modeled hemodynamic flow patterns in a heart with a septal shunt device.

FIG. 4B is a second schematic diagram illustrating modeled hemodynamic flow patterns in a heart with a septal shunt device.

FIG. 5A is a first schematic diagram illustrating modeled hemodynamic flow patterns in a heart with a left atrium to coronary sinus shunt device.

FIG. 5B is a second schematic diagram illustrating modeled hemodynamic flow patterns in a heart with a left atrium to coronary sinus shunt device.

FIG. 6 is a vector flow map of a benchtop model of a heart.

FIG. 7 is a vector flow map of a benchtop model of a heart with a septal shunt device.

FIG. 8 is a vector flow map of a benchtop model of a heart with a left atrium to coronary sinus shunt device.

FIG. 9 is a scatterplot of simulated cardiac output.

FIG. 10 is a scatterplot of simulated mean atrial pressure in a right atrium.

FIG. 11 is a scatterplot of simulated cardiac output.

FIG. 12 is a scatterplot of simulated mean ventricular pressure in a right ventricle.

FIG. 13 is a scatterplot of simulated mean atrial pressure in a right atrium.

FIG. 14A is a perspective view of a shunt device.

FIG. 14B is a side view of the shunt device.

FIG. 14C is a bottom view of the shunt device.

FIG. 15 is a perspective view of the shunt device in a collapsed configuration.

FIG. 16 is a side view of the shunt device with a sensor and anchored to a tissue wall.

FIG. 17 is a top view looking through an angled central flow tube of the shunt device.

FIG. 18 is a cross-sectional view through the heart showing the shunt device implanted in a tissue wall between a left atrium and a coronary sinus of the heart.

FIG. 19 is a cross-sectional view through the heart showing the shunt device implanted in the tissue wall and a flow path from the left atrium to a right atrium through the shunt device.

FIG. 20 is a flowchart showing a method for selecting a shunt device for implantation in the heart.

FIG. 21 is a schematic view of a shunt in a tissue wall between a coronary sinus and a left atrium.

FIG. 22 is a perspective view of an armless shunt device.

FIG. 23 is a side view of the armless shunt device of FIG. 22.

### DETAILED DESCRIPTION

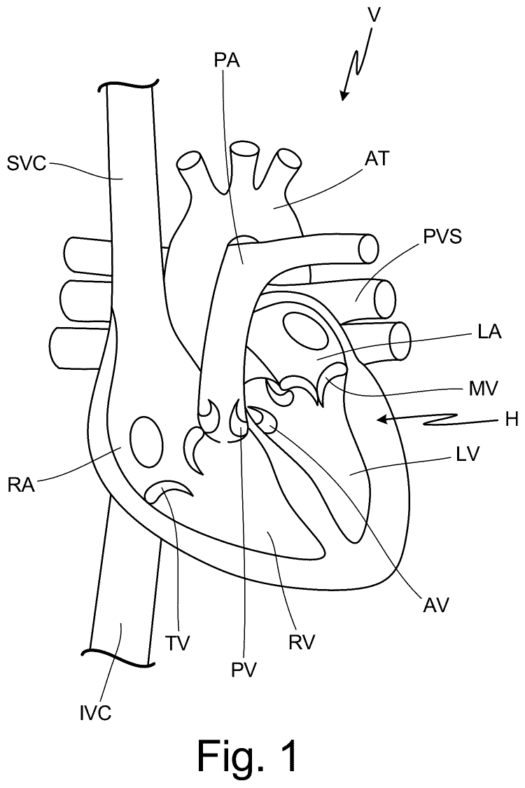

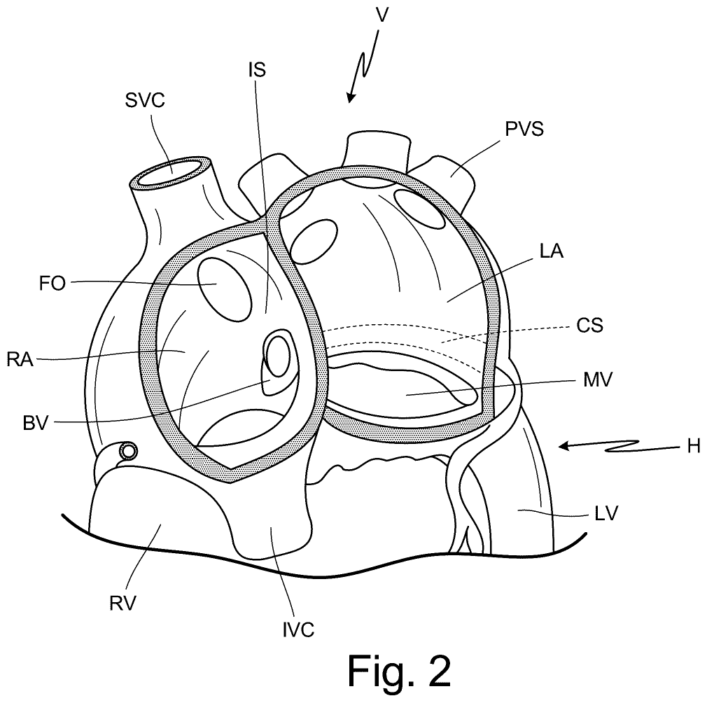

FIG. 1 is a schematic diagram of heart H and vasculature V. FIG. 2 is a cross-sectional schematic view of heart H. FIGS. 1-2 will be discussed together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC, inferior vena cava IVC, and coronary sinus CS.

A majority of the blood flows into right atrium RA from superior vena cava SVC and inferior vena cava IVC, which are offset from one another. Due to the offset of the major entry blood flows from superior vena cava SVC and inferior vena cava IVC, a natural flow vortex occurs in right atrium RA (a right-sided flow vortex). This allows a substantial portion of blood from right atrium RA to pass through right atrium RA and enter right ventricle RV by direct flow. The right-sided flow vortex in right atrium RA preserves kinetic energy and momentum of the major blood flows entering right atrium RA and allows a substantial portion of blood to naturally pass from right atrium RA to right ventricle RV without any contribution to flow needed from the pumping action of right atrium RA. With contraction, right atrium RA also pumps the residual portion of the entering blood not caught in the direct flow through tricuspid valve TV into right ventricle RV. The blood enters right ventricle RV and then flows through pulmonary valve PV into pulmonary artery PA. With preservation of direct inflow from right atrium RA, blood entering right ventricle RV also forms a natural flow vortex (a right-ventricular flow vortex) in right ventricle RV, which naturally re-directs blood entering right ventricle RV to pulmonary artery PA by direct flow without requiring right ventricle RV to perform substantial work of pumping blood. Residual blood that is not transported to pulmonary artery PA via pulmonary valve PV by direct flow is pumped by the contraction of right ventricle RV. The blood flows from pulmonary artery PA into smaller arteries that deliver the deoxygenated blood to the lungs via the pulmonary circulatory system. The lungs can then oxygenate the blood.

The left side of heart H, including left atrium LA and left ventricle LV, receives the oxygenated blood from the lungs and provides blood flow to the body. Blood flows into left atrium LA from pulmonary veins PVS. The offset of the right and left pulmonary veins PVS also leads to the formation of a natural flow vortex in left atrium LA (left-sided flow vortex), which helps maintain momentum and minimize work as the blood traverses left atrium LA to mitral valve MV. Direct flow, as described above, and the pumping action of left atrium LA propels the blood through mitral valve MV into left ventricle LV. As the blood enters left ventricle LV, a natural flow vortex (a left-ventricular flow vortex) forms in left ventricle LV, which redirects flow naturally towards the left ventricular outflow of aortic valve AV so that it can be efficiently pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it approaches right atrium RA. Coronary sinus CS also wraps around a portion of right atrium RA posteriorly before in enters right atrium RA via the ostium of coronary sinus CS lateral and posterior to an orifice of tricuspid valve TV, and medial to inferior vena cava IVC entry point. Due to its proximity to inferior vena cava IVC, blood entering right atrium RA from coronary sinus CS is naturally entrained into the larger inflow from inferior vena cava IVC forming the natural flow vortex (right-sided flow vortex) in right atrium RA, which naturally redirects the inflows towards tricuspid valve TV.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood, primarily from inferior vena cava IVC and coronary sinus CS, to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and enters the inferior vena cava IVC via a natural shunt called the ductus venosus. The oxygenated blood moves through inferior vena cava IVC to right atrium RA. The opening of inferior vena cava IVC in right atrium RA is positioned to direct the oxygenated blood through right atrium RA and then through a second natural shunt called foramen ovale into left atrium LA along with the entrained deoxygenated blood from coronary sinus CS. Left atrium LA can then pump the mixed oxygenated and deoxygenated blood into left ventricle LV, which pumps it to aorta AT and the systemic circulatory system. This allows the pulmonary circulatory system to be bypassed in utero. Some deoxygenated blood, primarily from superior vena cava SVC, is pumped through the right heart where it also bypasses the lungs and reenters aorta AT via a third natural shunt called the ductus arteriosus. Upon birth, respiration expands the lungs, blood begins to circulate through the lungs to be oxygenated, and the three natural shunts close. The closure of the foramen ovale forms fossa ovalis FS.

Shunt devices can be positioned in heart H to shunt blood between left atrium LA and right atrium RA. Left atrium LA has a higher pressure and lower compliance compared to right atrium RA, and right atrium RA has a lower pressure and higher compliance than left atrium LA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema. Reduction of back pressure on the pulmonary circulation also reduces pulmonary artery PA pressures, which can injure the small arteries leading to the lungs resulting in pulmonary hypertension. Increased pulmonary artery pressures can also lead to pressure overload of right ventricle RV, injuring right ventricle RV and potentially leading to right sided heart failure.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of left ventricle LV and certain forms of congenital heart and valve disease.

Septal shunt devices (also called inter-atrial shunt devices or trans-septal shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow primarily from right atrium RA to left atrium LA. This causes a risk of paradoxical stroke (also known as paradoxical embolism), as emboli can move from right atrium RA to left atrium LA via the relatively short flow path of the shunt and then through left atrium LA into aorta AT and the systemic circulation as a result of physiologic conditions that may cause temporary bidirectional flow at different times in the cardiac cycle.

Shunt devices can also be left atrium to coronary sinus shunt devices that are positioned in a tissue wall between left atrium LA and coronary sinus CS where the two structures are in close approximation as coronary sinus CS passes through the atrio-ventricular groove that is covered by epicardium. Left atrium to coronary sinus shunt devices move blood from left atrium LA into coronary sinus CS, which then delivers the blood to right atrium RA via the ostium of coronary sinus CS, the natural orifice of coronary sinus CS, which may have thebesian valve BV. Coronary sinus CS is compliant and can quickly grow in response to increased volume with conditions such as drainage of the left subclavian vein to coronary sinus CS. Similarly, coronary sinus CS can act as an additional compliance chamber when using a left atrium to coronary sinus shunt device. Left atrium to coronary sinus shunt devices may further provide increased protections against paradoxical strokes by increasing the length of the flow path blood must traverse to get from right atrium RA to left atrium LA, as the blood would have to flow retrograde from right atrium RA through a significant distance in coronary sinus CS before entering left atrium LA. Further, left atrium to coronary sinus shunt devices also provide protection against significant right atrium RA to left atrium LA shunting of fully deoxygenated blood as it would have to flow retrograde from right atrium RA through coronary sinus CS for a substantial distance before entering left atrium LA.

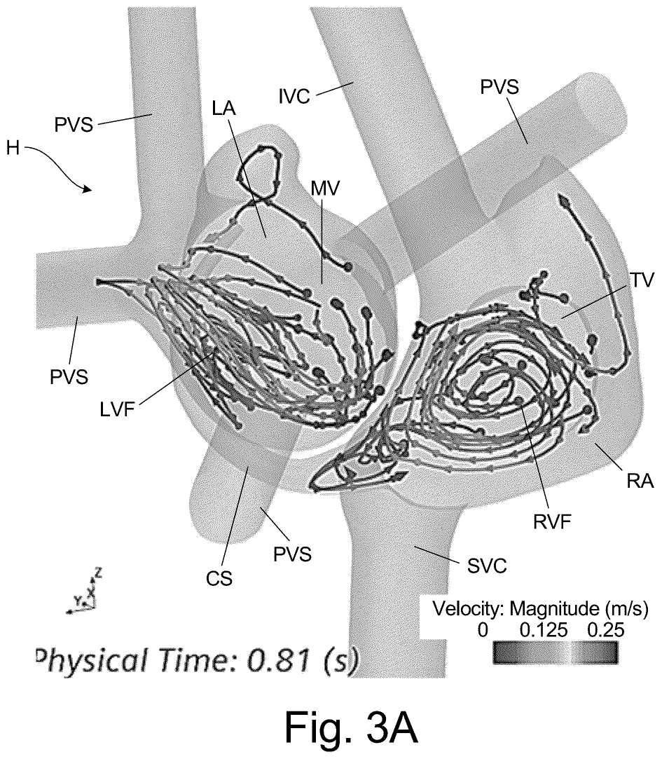

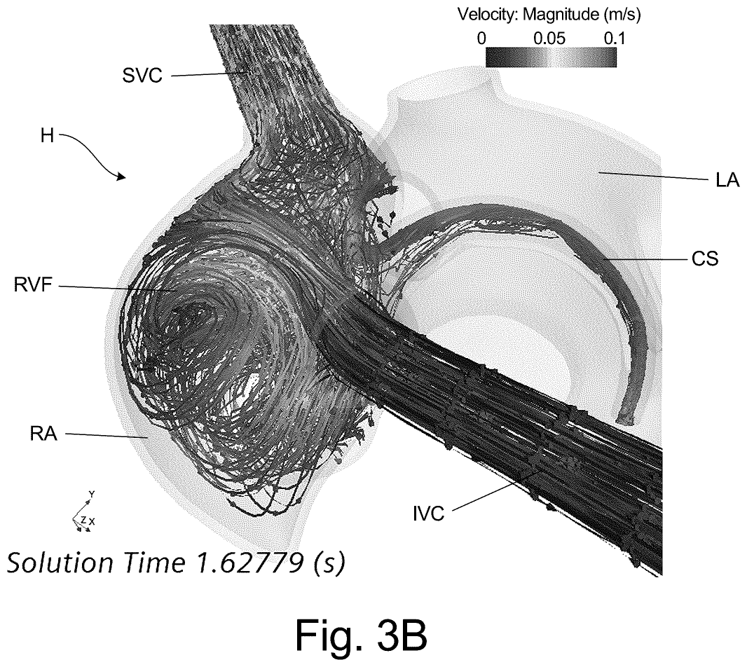

It has also been hypothesized that a left atrium to coronary sinus shunt device has a lesser disruption on the natural flow patterns of blood moving through left atrium LA, right atrium RA, and coronary sinus CS as compared to a traditional septal shunt device. Further, it is hypothesized that a left atrium to coronary sinus shunt device can enhance the natural vortical flow pattern of blood moving through right atrium RA, as the blood from coronary sinus CS is entrained into inferior vena cava IVC inflow. These flow patterns will be discussed below in greater detail with respect to FIGS. 3A-5B.

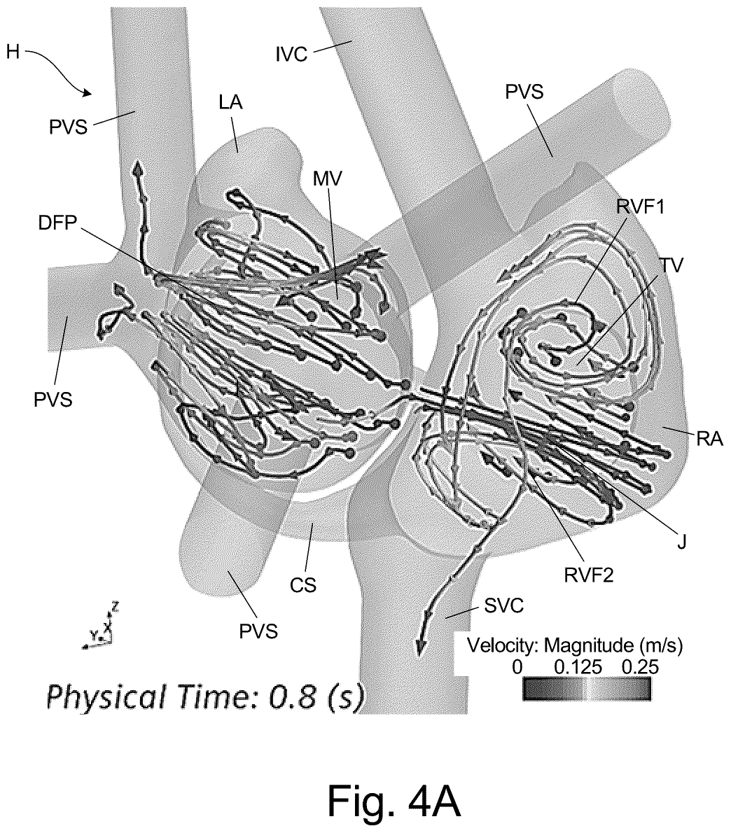

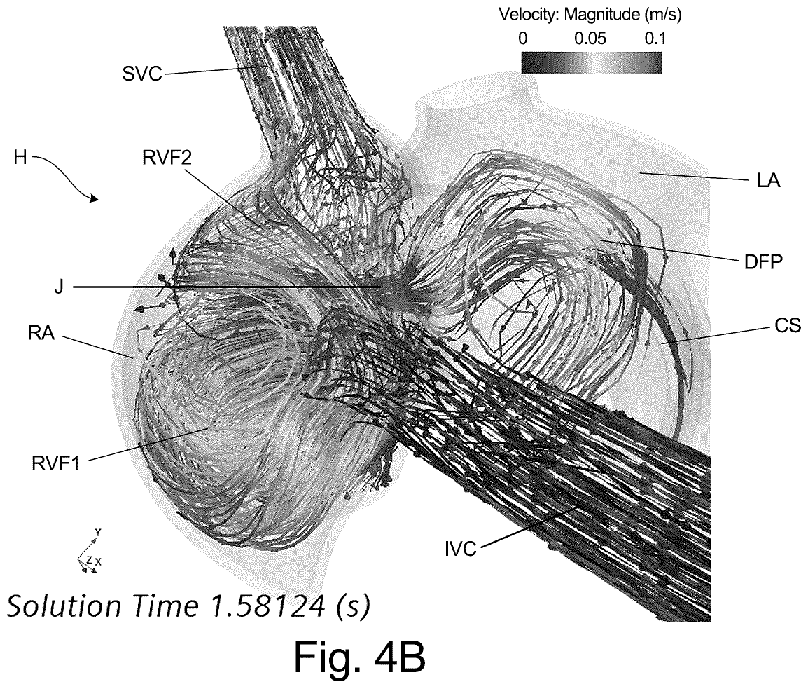

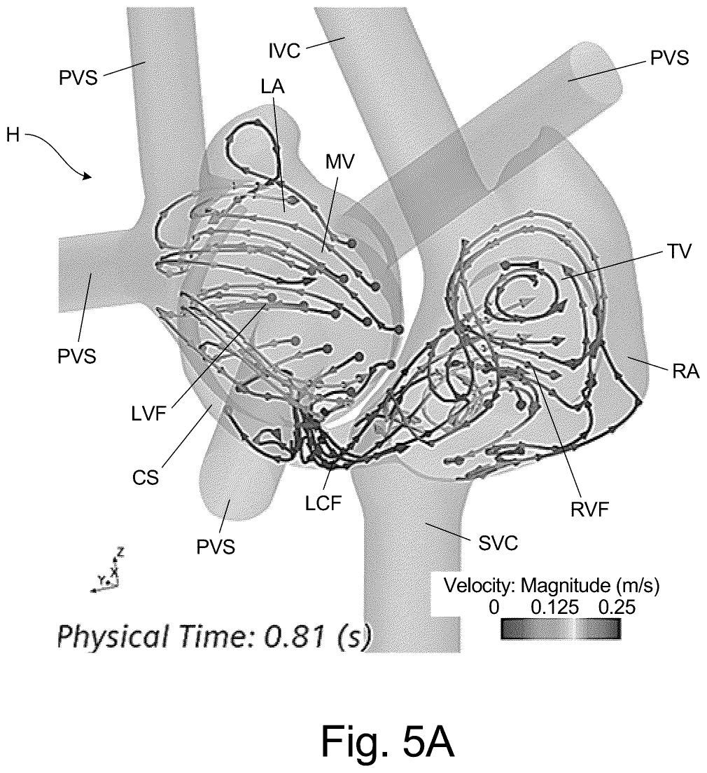

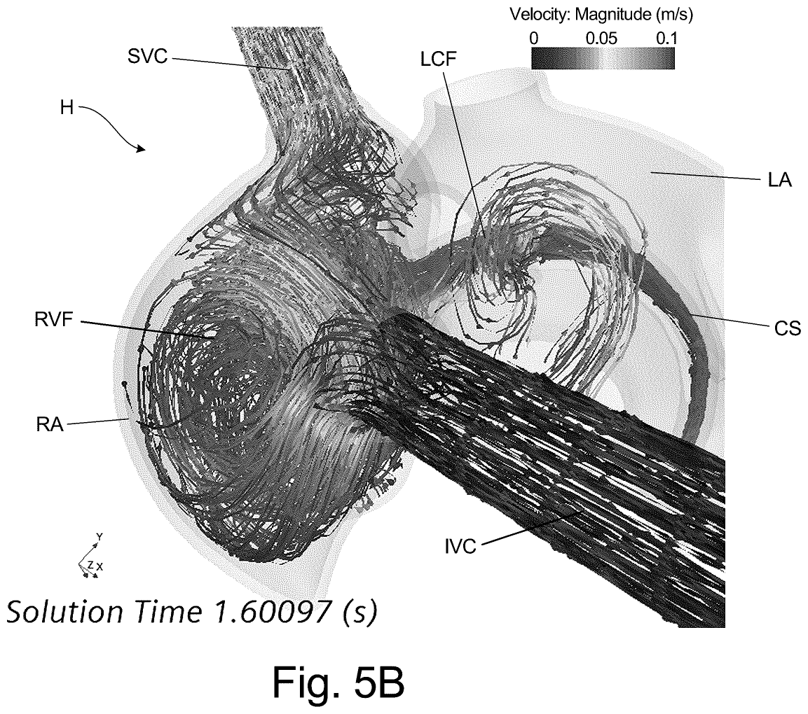

FIG. 3A is a first schematic diagram illustrating modeled hemodynamic flow patterns in heart H. FIG. 3B is a second schematic diagram illustrating modeled hemodynamic flow patterns in heart H. FIG. 4A is a first schematic diagram illustrating modeled hemodynamic flow patterns in heart H with a septal shunt device. FIG. 4B is a second schematic diagram illustrating modeled hemodynamic flow patterns in heart H with a septal shunt device. FIG. 5A is a schematic diagram illustrating modeled hemodynamic flow patterns in heart H with a left atrium to coronary sinus shunt device. FIG. 5B is a second schematic diagram illustrating modeled hemodynamic flow patterns in heart H with a left atrium to coronary sinus shunt device. FIGS. 3A-5B show heart H, right atrium RA, left atrium LA, superior vena cava SVC, inferior vena cava IVC, and coronary sinus CS. FIGS. 3A, 4A, and 5A show tricuspid valve TV, pulmonary veins PVS, and mitral valve MV.

FIGS. 3A-5B show modeled velocity stream lines representing hemodynamic flow patterns in heart H. FIGS. 3A, 4A, and 5A show heart H oriented with right atrium RA on a right side of the figures and left atrium LA on a left side of the figures. FIGS. 3A, 4A, and 5A are inferior views of heart H. FIGS. 3B, 4B, and 5B show heart H oriented with right atrium RA on a left side of the figures and left atrium LA on a right side of the figures. FIGS. 3B, 4B, and 5B are superior views of heart H.

Natural flow patterns of blood flow exist in heart H and help move blood through heart H and into the vasculature connected to heart H in a way that maximizes preservation of blood flow momentum and kinetic energy. The natural flow pattern for blood moving through arteries and veins is typically helical in nature (helical flow patterns). The natural flow pattern for blood moving through the chambers of heart H is typically vortical in nature (vortical flow patterns).

FIG. 3A shows modeled hemodynamic flow patterns that exist in right atrium RA and left atrium LA of heart H. FIG. 3B shows modeled hemodynamic flow patterns that exist in right atrium RA, superior vena cava SVC, inferior vena cava IVC, and coronary sinus CS. FIGS. 3A-3B represent natural flow patterns that are formed in heart H, including right atrium RA and left atrium LA, based on the offset inflows of blood into the chambers of heart H in addition to the anatomical structure of heart H. When looking at heart H from the right side (the right sagittal view), a clockwise right-sided flow vortex is formed in right atrium RA and a counter-clockwise left-sided flow vortex is formed in left atrium LA. The right-sided flow vortex in right atrium RA is the natural flow pattern of blood flow in right atrium RA. The left-sided flow vortex in left atrium LA is the natural flow pattern of blood flow in left atrium LA. The modeled hemodynamic flow patterns shown in FIGS. 3A-3B represent intra-cardiac flow patterns for a structurally normal heart.

Blood flows enter the right atrium RA from superior vena cava SVC, inferior vena cava IVC, and coronary sinus CS. The superior vena cava opening and the inferior vena cava opening in right atrium RA are offset so that the blood flowing into right atrium RA from superior vena cava SVC and inferior vena cava IVC do not collide with each other. Due to its orientation and physical proximity, coronary sinus CS flow is entrained into inferior vena cava IVC flow. The blood flowing through superior vena cava SVC and inferior vena cava IVC has a helical flow pattern. A majority of the blood in right atrium RA enters right atrium RA through inferior vena cava IVC, and the blood flowing from inferior vena cava IVC into right atrium RA is pointed towards the top of right atrium RA. The helical flow pattern of the blood flowing into right atrium RA from inferior vena cava IVC helps to form a clockwise right-sided flow vortex in right atrium RA (when looking at the heart from the right side). The flow of blood entering right atrium RA from superior vena cava SVC will flow along the inter-atrial septum and towards tricuspid valve TV. The helical flow pattern of the blood flowing from superior vena cava SVC into right atrium RA helps the flow of blood naturally join with the clockwise right-sided flow vortex formed in right atrium RA by the flow of blood from inferior vena cava IVC, which is joined by coronary sinus CS flow. A small amount of blood flows into right atrium RA from coronary sinus CS. The flow flowing through coronary sinus CS will have a helical flow pattern. The helical flow pattern of the blood exiting coronary sinus CS will naturally join with inferior vena cava IVC flow and the right-sided flow vortex in right atrium RA. The right-sided flow vortex in right atrium RA is shown with velocity stream lines labeled RVF in FIGS. 3A-3B.

The right-sided flow vortex formed in right atrium RA helps the blood flow through right atrium RA, through tricuspid valve TV, into the right ventricle, through the pulmonary valve, and into the pulmonary artery. The right heart is an inefficient pump and can act more like a conduit. The right-sided flow vortex formed in the right heart helps to preserve kinetic energy and the momentum of blood flow as it moves from superior vena cava SVC and inferior vena cava IVC (the Vena Cavae) through the right heart and into the pulmonary artery, even with minimal to no pumping being provided by the right heart. This is especially important for maintaining right heart output, which must match left heart output, during periods of high output and heart rates during exercise. The right-sided flow vortex formed in right atrium RA helps to move the blood from right atrium RA through tricuspid valve TV and into the right ventricle with minimal loss of momentum and kinetic energy. The blood shoots from right atrium RA through the right ventricle, out the right ventricular outflow tract, through the pulmonary valve, and into the pulmonary artery. Approximately 50% of the blood will flow into the pulmonary artery without any pumping required by the right heart because of the right-sided flow vortices of right atrium RA and right ventricle RV and anatomical constraints of the right heart. Right heart contraction enhances the flow of residual blood through the right heart.

Blood flows into left atrium LA from pulmonary veins PVS. There are four pulmonary veins PVS that flow into left atrium LA. The blood flowing through pulmonary veins PVS has a helical flow pattern. The offset of helical flow of the blood flowing from pulmonary veins PVS into left atrium LA helps to form a counter-clockwise left-sided flow vortex (when looking at the heart from the right side) in left atrium LA. The left-sided flow vortex in left atrium LA directs flow towards mitral valve MV. The left-sided flow vortex in left atrium LA is shown with velocity stream lines labeled LVF in FIG. 3A.

It is hypothesized that if the intra-cardiac blood flow patterns in heart H (including right-sided flow vortex in right atrium RA and left-sided flow vortex in left atrium LA) are disrupted, blood flow from superior vena cava SVC and inferior vena cava IVC (the Vena Cavae), through right atrium RA, through the right ventricle, and into the pulmonary artery, and blood flow from the pulmonary veins, through the left atrium LA, through the left ventricle, and into the aorta become less efficient and place increased mechanical workloads on the respective ventricles. This is especially important in already failing hearts, where the ability to increase the workload of the heart muscle is impaired. Disruptions in the intra-cardiac blood flow patterns in heart H (including right-sided flow vortex in right atrium RA and left-sided flow vortex in left atrium LA) can happen for a variety of reasons. For example, the anatomy of heart H can change as patients age. This can affect the offset between the opening of superior vena cava SVC and the opening of inferior vena cava IVC. The blood flow entering right atrium RA from superior vena cava SVC and the blood flow entering right atrium RA from inferior vena cava IVC can collide as the anatomy of heart H changes, which disrupts the natural formation of the right-sided flow vortex in right atrium RA. In another example, right atrium RA can be enlarged in patients with heart failure with or without atrial fibrillation. The enlargement of right atrium RA can also disrupt the right-sided flow vortex formed in right atrium RA. Similarly, left atrium LA can be enlarged in patients with heart failure with or without atrial fibrillation. The enlargement of left atrium LA can disrupt the left-sided flow vortex formed in left atrium LA. Additionally, patients with a patent foramen ovale (a natural inter-atrial septal shunt) or a secundum atrial septal defect due to failure of the patent foramen ovale to fully close may not have the expected intra-cardiac blood flow patterns (including right-sided flow vortex in right atrium RA and left-sided flow vortex in left atrium LA), including the expected flow vortexes created during atrial filling. Closure of a secundum atrial septal defect with altered right atrial non-single vortex flow patterns has been shown to revert to a dominant single vortical flow pattern after the atrial septal defect is occluded.

When the right-sided flow vortex in right atrium RA changes, momentum and energy of the blood flow are lost and the right heart needs to pump harder to move the blood from right atrium RA into the right ventricle and the pulmonary artery. This is due to the right-sided flow vortex contributing less to the movement of blood through the right heart. Similarly, when the left-sided flow vortex in left atrium LA changes, the left heart needs to pump harder to move the blood from left atrium LA into the left ventricle and the aorta. This is due to the left-sided flow vortex contributing less to the movement of blood through the left heart. Further, as the intra-cardiac flow patterns in heart H (including right-sided flow vortex in right atrium RA and left-sided flow vortex in left atrium LA) change due to age or disease, areas of turbulence can be created in the flow patterns of heart H and there can be a loss of fluid dynamics leading to inefficiencies that could lead to diminished flow. This can increase the susceptibility of the right heart and/or the left heart to fail (the inability to pump enough blood to meet the body's oxygen demands), as heart H has to do more work to move the same amount of blood through heart H. More work is needed to recreate the lost momentum naturally preserved by the intra-cardiac flow patterns in heart H (including right-sided flow vortex in right atrium RA and left-sided flow vortex in left atrium LA), putting additional strain on heart H.

Changes in intra-cardiac flow patterns change intra-cardiac energetics. Heart H is uniquely designed to maximize efficiency by preserving the kinetic energy and momentum of blood flow, thus minimizing the work needed to propagate the blood flow into the chambers, between the chambers, and out of the chambers. Anything that disrupts the intra-cardiac flow patterns in heart H (including right-sided flow vortex in right atrium RA and left-sided flow vortex in left atrium LA) can reduce the efficiency of the energetics of heart H due to a loss of potential energy, which makes it more difficult for heart H to do its job of propagating blood into, between, and out of the chambers. Anything that disrupts the intra-cardiac flow patterns through heart H (including right-sided flow vortex in right atrium RA and left-sided flow vortex in left atrium LA) can increase the amount of work heart H has to do, prolong transit times through heart H, and makes it more difficult for heart H to eject blood. This is especially problematic for people experiencing heart failure, as the heart failure can be exacerbated due to disruptions in the intra-cardiac flow patterns through heart H (including right-sided flow vortex in right atrium RA and left-sided flow vortex in left atrium LA).

FIG. 4A shows modeled hemodynamic flow patterns that exist in right atrium RA and left atrium LA of heart H when a septal shunt device is positioned between right atrium RA and left atrium LA. FIG. 4B shows modeled hemodynamic flow patterns that exist in right atrium RA, superior vena cava SVC, inferior vena cava IVC, coronary sinus CS, and left atrium LA when a septal shunt device is positioned between right atrium RA and left atrium LA. A septal shunt device has been modeled in the inter-atrial septum between right atrium RA and left atrium LA in the schematic shown in FIGS. 4A-4B to allow blood to shunt directly from left atrium LA to right atrium RA.

As shown in FIGS. 4A-4B, when a septal shunt device is positioned in the inter-atrial septum between right atrium RA and left atrium LA, blood jets from left atrium LA into and across right atrium RA. The jet of blood is shown with velocity stream lines labeled J in FIGS. 4A-4B. The jet of blood in right atrium RA disrupts the right-sided flow vortex in right atrium RA. When the blood jets across right atrium RA, two separate flow vortexes are formed in right atrium RA. The first flow vortex is shown with velocity stream lines labeled RVF1, and the second flow vortex is shown with velocity stream lines labeled RVF2 in FIGS. 4A-4B. There is also a disruption of the left-sided flow vortex in left atrium LA. The septal shunt device is not aligned with the left-sided flow vortex in left atrium LA, but the pressure difference between right atrium RA and left atrium LA causes the blood in left atrium LA to move out of the left-sided flow vortex and through the septal shunt device into right atrium RA. The disrupted left-sided flow vortex in left atrium LA is shown with velocity stream lines labeled DFP in FIGS. 4A-4B. This disruption of the right-sided flow vortex in right atrium RA and the left-sided flow vortex in left atrium LA can also lead to loss of right ventricle RV and left ventricle LV vortex formations and will cause heart H to have to work harder to pump blood through their respective ventricles and can lead to the development or worsening of heart failure over time.

Specifically, when looking at the right heart, a septal shunt device introduces a significant disruption to the right-sided flow vortex in right atrium RA as the blood jets across right atrium RA. It is hypothesized that the disruption to the right-sided flow vortex in right atrium RA can cause or exacerbate right heart failure. Disruption of the right-sided flow vortex in right atrium RA means that the momentum and kinetic energy of blood naturally or efficiently flowing from right atrium RA into the right ventricle and the pulmonary artery is lost. In order to move the blood from right atrium RA into the right ventricle and the pulmonary artery, the right heart has to work harder to pump the blood. This increased work required by the right heart can cause or exacerbate right heart failure and places a severe load on the less efficient right heart during periods of exercise, where heart rates are high and diastolic filling periods are short.

FIG. 5A shows modeled hemodynamic flow patterns that exist in right atrium RA and left atrium LA of heart H when a left atrium to coronary sinus shunt device is positioned in a tissue wall between left atrium LA and coronary sinus CS. FIG. 5B shows modeled hemodynamic flow patterns that exist in right atrium RA, superior vena cava SVC, inferior vena cava IVC, coronary sinus CS, and left atrium LA when a left atrium to coronary sinus shunt device is positioned in a tissue wall between left atrium LA and coronary sinus CS. A shunt device has been modeled in the tissue wall between left atrium LA and coronary sinus CS in the schematics shown in FIGS. 5A-5B to allow blood to shunt from left atrium LA to coronary sinus CS, which then delivers the blood to right atrium RA.

As shown in FIGS. 5A-5B, when a left atrium to coronary sinus shunt device is positioned in the tissue wall between left atrium LA and coronary sinus CS, blood moves from left atrium LA into coronary sinus CS. The blood moving from left atrium LA to coronary sinus CS in shown with velocity stream lines labeled LCF in FIGS. 5A-5B. The blood moving from left atrium LA to coronary sinus CS minimizes disruption to the flow of blood in left atrium LA and coronary sinus CS. The blood moves from left atrium LA to coronary sinus CS at a high velocity due to the pressure difference between left atrium LA and coronary sinus CS. Positioning a shunt device in the tissue wall between left atrium LA and coronary sinus CS close to the mitral annulus positions the shunt device to guide blood from the left-sided flow vortex in left atrium LA through the shunt device to join with the flow pattern in coronary sinus CS. As blood flows in the left-sided flow vortex in left atrium LA (shown with velocity stream lines labeled LVF in FIG. 3C), it flows against the tissue wall where the left atrium to coronary sinus shunt device is placed and will naturally flow through the shunt device. This minimizes the disruption to the left-sided flow vortex in left atrium LA. Further, the blood that flows through the left atrium to coronary sinus shunt device joins into the helical flow of blood flowing through coronary sinus CS. This minimizes disruptions to the flow of blood through coronary sinus CS. The pressure of the blood in coronary sinus CS will increase due to the increase in volume of blood in coronary sinus CS. Further, coronary sinus CS can dilate due to the increased volume of blood in coronary sinus CS.

As blood normally flows from coronary sinus CS into right atrium RA where it is entrained into the flow path of inferior vena cava IVC, there is minimal to no disruption of the right-sided flow vortex in right atrium RA (shown with velocity stream lines labeled RVF in FIGS. 5A-5B). The modeled flow patterns shown in FIGS. 5A-5B show less disruption of the right-sided flow vortex in right atrium RA as compared to the modeled flow patterns when using a septal shunt device as shown in FIGS. 4A-4B. The minimal to no disruption of the right-sided flow vortex in right atrium RA with a left atrium to coronary sinus shunt reduces energy loss in right atrium RA, thus requiring less work from the right heart to pump blood through the right atrium to the right ventricle and the pulmonary artery. A left atrium to coronary sinus shunt device can provide fluidic and dynamic advantages that have the potential to avoid disruptions in asymmetric redirection of streaming blood flow through the right heart. Further, it can preserve the right-sided flow vortex that arises in right atrium RA during the atrial filling stage, which may transport the momentum efficiently to reduce energy loss.

It is hypothesized that the increased velocity of the flow of blood entering right atrium RA from coronary sinus CS when using a left atrium to coronary sinus shunt device enhances and/or augments the right-sided flow vortex formed in right atrium RA. The increased velocity of the flow of blood entering right atrium RA from coronary sinus CS when using a left atrium to coronary sinus shunt device can help to reestablish or reinforce the right-sided flow vortex in right atrium RA for patients who have lost their right-sided flow vortex due to age and/or disease. The flow from coronary sinus CS exits the ostium of coronary sinus CS, flows upwards in right atrium RA and as it is entrained into the inferior vena cava flow, may augment and/or reestablish the vortical flow pattern in right atrium RA. Alternatively, the now enhanced flow from coronary sinus CS, which in the presence of a shunt device increases to 20-40% of cardiac output, may direct blood through tricuspid valve TV in a path that preferentially shoots blood out the right ventricle outflow tract to the pulmonary artery. A left atrium to coronary sinus shunt device can preserve, and may even enhance, the momentum of inflowing blood streams being redirected towards atrio-ventricular valves of heart H. Further, it can preserve, and may even enhance, the normal exit of blood flow from right atrium RA to the right ventricle, which promotes change in flow direction at a ventricular level such that recoil away from ejected blood is in a direction that can enhance rather than inhibit ventriculo-atrial coupling.

A left atrium to coronary sinus shunt device takes advantage of normal flow paths in heart H and minimizes dissipative interaction between entering, recirculating, and outflowing blood streams. Recirculating flows and vortices are characteristic flow features in between cardiac chambers, which play a crucial role in momentum transfer and irreversible energy loss. A left atrium to coronary sinus shunt device preserves the natural intra-atrial and intra-ventricular flow structures of the healthy human heart that are optimal for minimizing energy dissipation. An increase in energy dissipation due to the break of the natural flow structure may lead to an increase in the energy that is needed to be generated by myocardial muscle to eject the blood into the circulation (increases myocardial work in an already pressure overloaded ventricle).

A left atrium to coronary sinus shunt device does not interfere with the reciprocating, sling-like “morphodynamic” mode of action that comes into effect when heart rate and output increases during exercise. It is hypothesized that a left atrium to coronary sinus shunt device has potential functional advantages that could gain importance as flow velocities, heart rate and rates of change of momentum increase with exertion. A left atrium to coronary sinus shunt device does not detract from the ability of the looped heart to deliver enhanced output during strenuous exertion. Rather it will enhance responses to exercise, as the looped heart is able to function “morphodynamically,” redirecting and slinging blood through its sinuous curvatures with minimal dissipation of energy and with dynamically enhanced reciprocation of atrial and ventricular function.

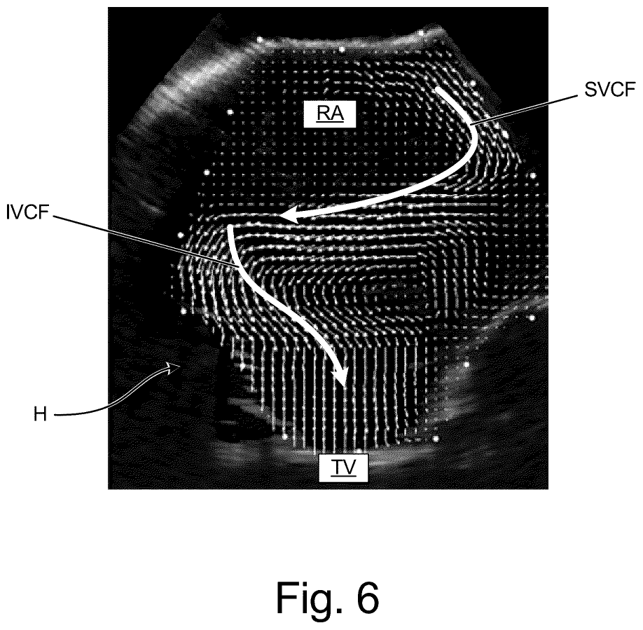

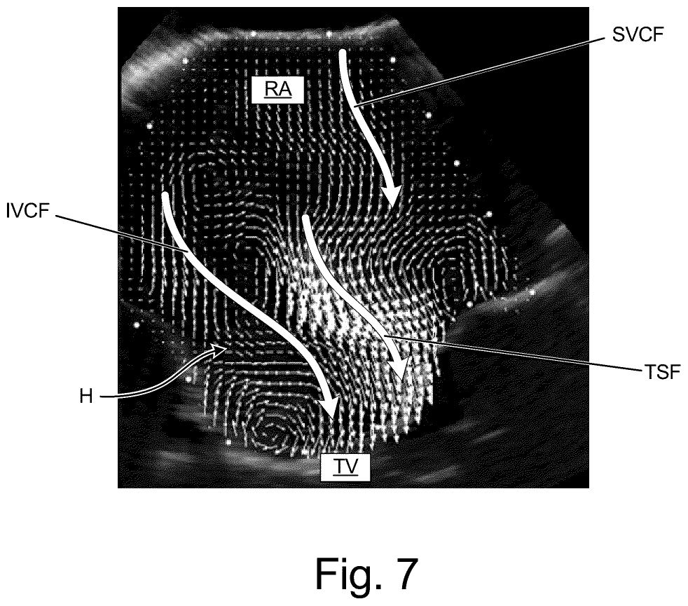

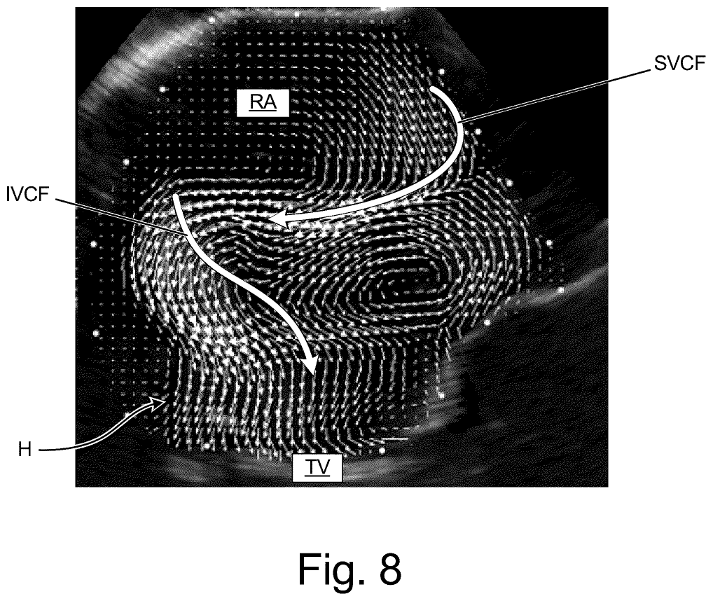

FIG. 6 is a vector flow map of a benchtop model of heart H. FIG. 7 is a vector flow map of a benchtop model of heart H with a septal shunt device. FIG. 8 is a vector flow map of a benchtop model of heart H with a left atrium to coronary sinus shunt device. FIGS. 6-8 show heart H, right atrium RA, tricuspid valve TV, superior vena cava flow SVCF, and inferior vena cava flow IVCF. FIG. 7 further shows septal flow TSF.

A benchtop model of heart H was used to generate the vector flow maps shown in FIGS. 6-8. The benchtop model of heart H was capable of tuning to different heartrate and pressure conditions. The vector flow map shown in FIG. 6 shows the benchtop model of heart H in its natural state (i.e., without a shunt device). The vector flow map shown in FIG. 7 shows the benchtop model of heart H with a septal shunt device. The vector flow map shown in FIG. 8 shows the benchtop model of heart H with a left atrium to coronary sinus shunt device.

The vector flow maps shown in FIGS. 6-8 include vectors that represent the flow of blood through right atrium RA of heart H. In the vector flow maps shown in FIGS. 6-8, the higher intensity of vectors represents a higher velocity of blood flow through heart H. FIGS. 6-8 show inferior vena cava flow IVCF, which is the flow of blood through right atrium RA from the inferior vena cava and the coronary sinus. The coronary sinus flow is entrained in inferior vena cava flow IVCF. FIGS. 6-8 also show superior vena cava flow SVCF, which is the flow of blood through right atrium RA from the superior vena cava. Inferior vena cava flow IVCF and superior vena cava flow SVCF move towards tricuspid valve TV.

FIG. 6 shows heart H with the natural right-side flow vortex. As shown in FIG. 6, there is a slight intensity of vectors along superior vena cava flow SVCF and inferior vena cava flow IVCF. FIG. 7 shows heart H with a septal shunt device. As shown in FIG. 7, heart H with a septal shunt device has an intensity of vectors labeled TSF in FIG. 7. The intensity of vectors labeled TSF represents a flow of blood through the septal shunt device (septal flow TSF). Septal flow TSF has a high intensity of vectors. Septal flow TSF disrupts superior vena cava flow SVCF and inferior vena cava flow IVCF, as shown in FIG. 7. FIG. 8 shows heart H with a left atrium to coronary sinus shunt device. As shown in FIG. 8, heart H with a left atrium to coronary sinus shunt device has a greater intensity of vectors along superior vena cava flow SVCF and inferior vena cava flow IVCF indicating there is an increased velocity along superior vena cava flow SVCF and inferior vena cava flow IVCF. There is no disruption to superior vena cava flow SVCF or inferior vena cava flow IVCF.

Comparing FIG. 7 (heart H with a septal shunt device) to FIG. 8 (heart H with a left atrium to coronary sinus shunt device), it can be seen that a heart with a septal shunt device disrupts the natural flow of blood through heart H, namely superior vena cava flow SVCF and inferior vena cava flow IVCF. In comparison, heart H with a left atrium to coronary sinus shunt device has a greater intensity of vectors along superior vena cava flow SVCF and inferior vena cava flow IVCF when compared to the natural right-sided flow vortex through right atrium RA (see FIG. 6), however there is no disruption to superior vena cava flow SVCF and inferior vena cava flow IVCF.

The vector flow maps based on the benchtop model of heart H shown in FIGS. 6-8 confirm the simulated blood flows shown in FIGS. 3A-5B. As shown in FIGS. 6-8, a left atrium to coronary sinus shunt device minimizes the disruptions to the right-sided flow vortex in right atrium RA of heart H. As discussed above in reference to FIGS. 3A-5B, minimizing the disruptions of the right-sided flow vortex in right atrium RA of heart H reduces energy loss in right atrium and reduces the amount of work needed from the right heart to pump blood through heart H.

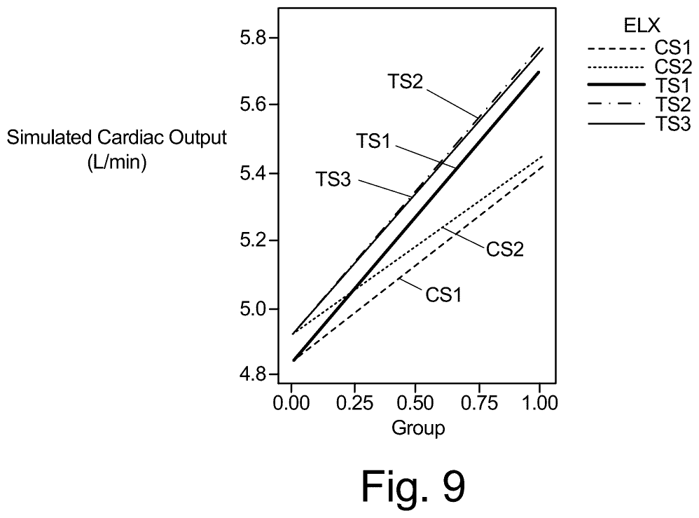

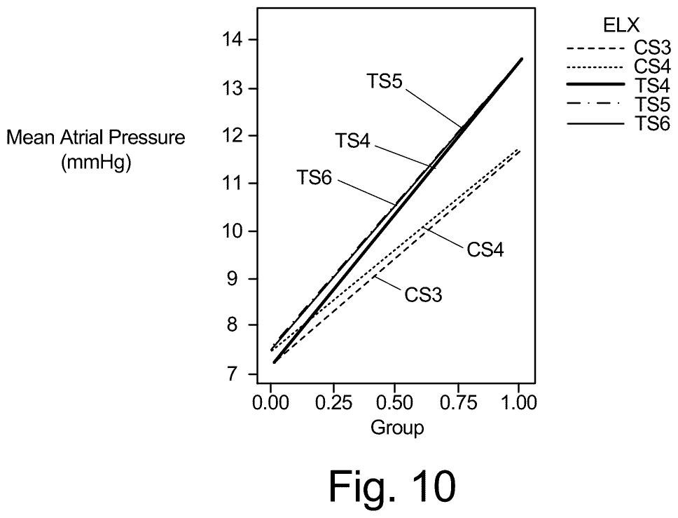

FIG. 9 is a scatterplot of simulated cardiac output. FIG. 10 is a scatterplot of simulated mean atrial pressure in a right atrium. FIGS. 9-10 will be discussed together. FIG. 9 shows trans-septal line TS1, trans-septal line TS2, trans-septal line TS3, coronary sinus line CS1, and coronary sinus line CS2. FIG. 10 shows trans-septal line TS4, trans-septal line TS5, trans-septal line TS6, coronary sinus line CS3, and coronary sinus line CS4.

A rigid benchtop model was used to generate the data for the scatterplots shown in FIGS. 9-10. The left side of the scatterplots show data collected before a shunt device was used (0.00 on the x-axis), and the right side of the scatterplots show data collected after a shunt device (a trans-septal shunt device or a left atrium to coronary sinus shunt device) was used (1.00 on the x-axis) in the rigid benchtop model.

FIG. 9 shows simulated cardiac output in liters per minute (L/min) on the y-axis. FIG. 9 shows trans-septal line TS1, trans-septal line TS2, trans-septal line TS3, coronary sinus line CS1, and coronary sinus line CS2. Trans-septal line TS1, trans-septal line TS2, and trans-septal line TS3 represent data that was gathered from a rigid benchtop model that received a trans-septal shunt device. Coronary sinus line CS1 and coronary sinus line CS2 represent data that was gathered from a rigid benchtop model that received a left atrium to coronary sinus shunt device.

FIG. 10 shows simulated mean atrial pressure in millimeters of Mercury (mmHg) on the y-axis. FIG. 10 shows trans-septal line TS4, trans-septal line TS5, trans-septal line TS6, coronary sinus line CS3, and coronary sinus line CS4. Trans-septal line TS4, trans-septal line TS5, and trans-septal line TS6 represent data that was gathered from a benchtop model that received a trans-septal shunt device. Coronary sinus line CS3 and coronary sinus line CS4 represent data that was gathered from a benchtop model that received a left atrium to coronary sinus shunt device.

As shown in FIG. 9, the simulated cardiac output was greater when a trans-septal shunt device was used compared to when a left atrium to coronary sinus shunt device was used. Further, as shown in FIG. 10, the mean atrial pressure in the right atrium increased after a shunt was implanted in the rigid benchtop model. As shown in FIG. 10, the mean atrial pressure in the right atrium was greater when a trans-septal shunt device was used compared to when a left atrium to coronary sinus shunt device was used.

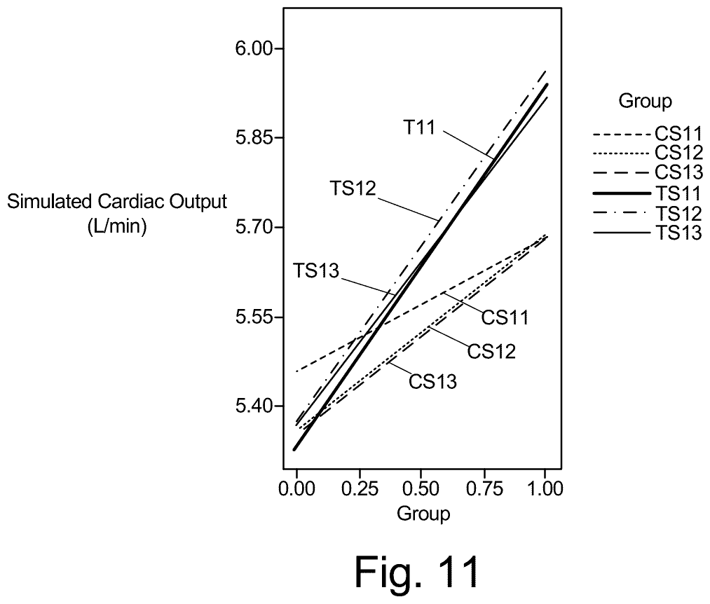

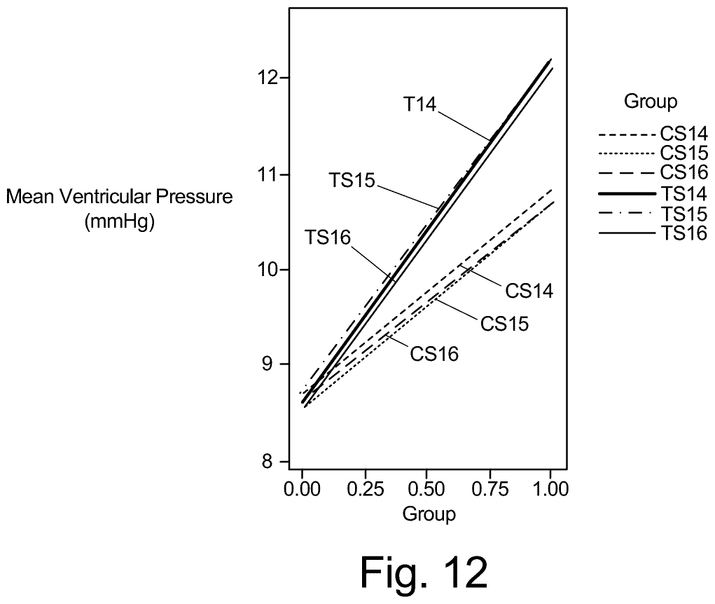

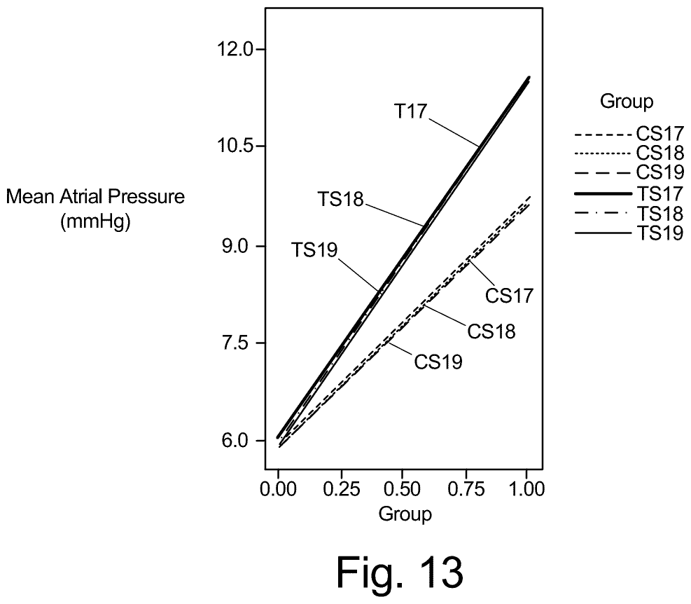

FIG. 11 is a scatterplot of simulated cardiac output. FIG. 12 is a scatterplot of simulated mean ventricular pressure in a right ventricle. FIG. 13 is a scatterplot of simulated mean atrial pressure in a right atrium. FIGS. 11-13 will be discussed together. FIG. 11 shows trans-septal line TS11, trans-septal line TS12, trans-septal line TS13, coronary sinus line CS11, coronary sinus line CS12, and coronary sinus line CS13. FIG. 12 shows trans-septal line TS14, trans-septal line TS15, trans-septal line TS16, coronary sinus line CS14, coronary sinus line CS15, and coronary sinus line CS16. FIG. 13 shows trans-septal line TS17, trans-septal line TS18, trans-septal line TS19, coronary sinus line CS17, coronary sinus line CS18, and coronary sinus line CS19.

A compliant benchtop model was used to generate the data for the scatterplots shown in FIGS. 11-13. The left side of the scatterplots show data collected before a shunt device was used (0.00 on the x-axis), and the right side of the scatterplots show data collected after a shunt device (a trans-septal shunt device or a left atrium to coronary sinus shunt device) was used (1.00 on the x-axis) in the compliant benchtop model.

FIG. 11 shows simulated cardiac output in liters per minute (L/min) on the y-axis. FIG. 11 shows trans-septal line TS11, trans-septal line TS12, trans-septal line TS13, coronary sinus line CS11, coronary sinus line CS12, and coronary sinus line CS13. Trans-septal line TS11, trans-septal line TS12, and trans-septal line TS13 represent data that was gathered from a compliant benchtop model that received a trans-septal shunt device. Coronary sinus line CS11, coronary sinus line CS12, and coronary sinus line CS13 represent data that was gathered from a compliant benchtop model that received a left atrium to coronary sinus shunt device.

FIG. 12 shows simulated mean ventricular pressure in millimeters of Mercury (mmHg) on the y-axis. FIG. 12 shows trans-septal line TS14, trans-septal line TS15, trans-septal line TS16, coronary sinus line CS14, coronary sinus line CS15, and coronary sinus line CS16. Trans-septal line TS14, trans-septal line TS15, and trans-septal line TS16 represent data that was gathered from a compliant benchtop model that received a trans-septal shunt device. Coronary sinus line CS14, coronary sinus line CS15, and coronary sinus line CS16 represent data that was gathered from a compliant benchtop model that received a left atrium to coronary sinus shunt device.

FIG. 13 shows simulated mean atrial pressure in millimeters of Mercury (mmHg) on the y-axis. FIG. 13 shows trans-septal line TS17, trans-septal line TS18, trans-septal line TS19, coronary sinus line CS17, coronary sinus line CS18, and coronary sinus line CS19. Trans-septal line TS17, trans-septal line TS18, and trans-septal line TS19 represent data that was gathered from a compliant benchtop model that received a trans-septal shunt device. Coronary sinus line CS17, coronary sinus line CS18, and coronary sinus line CS19 represent data that was gathered from a compliant benchtop model that received a left atrium to coronary sinus shunt device.

As shown in FIG. 11, the simulated cardiac output was greater when a trans-septal shunt device was used compared to when a left atrium to coronary sinus shunt device was used. Further, as shown in FIGS. 12-13, the mean ventricular pressure in the right ventricle and the mean atrial pressure in the right atrium increased after a shunt was implanted in the compliant benchtop model. As shown in FIGS. 12-13, the mean ventricular pressure in the right ventricle and the mean atrial pressure in the right atrium were greater when a trans-septal shunt device was used compared to when a left atrium to coronary sinus shunt device was used.

As shown in FIGS. 9-13, both rigid and compliant benchtop models demonstrate lower mean atrial and ventricular pressures and lower simulated cardiac output when a left atrium to coronary sinus shunt device is used compared to a trans-septal shunt device.

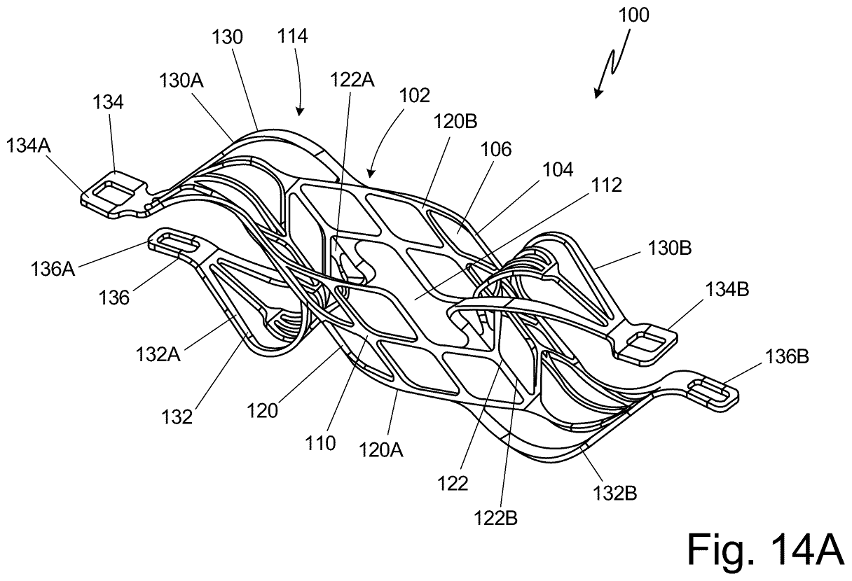

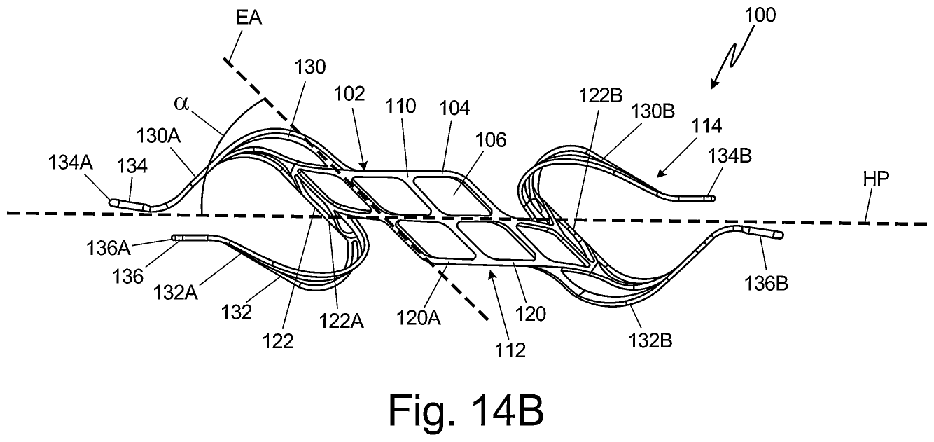

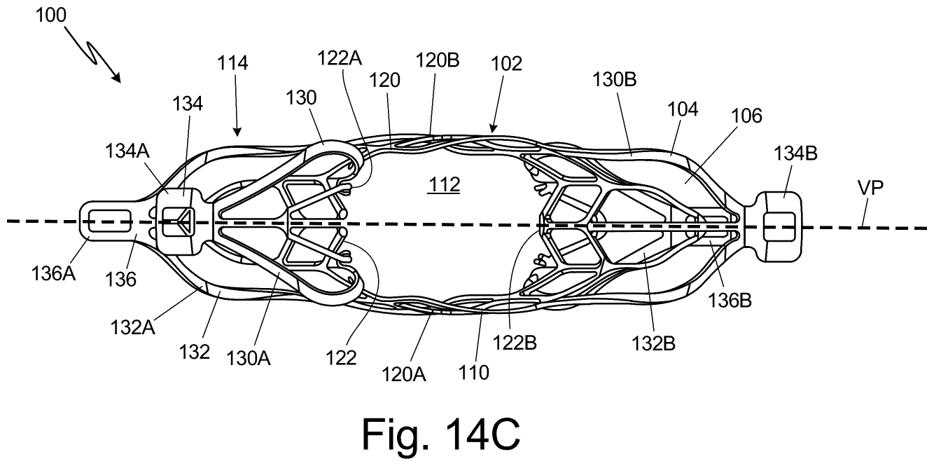

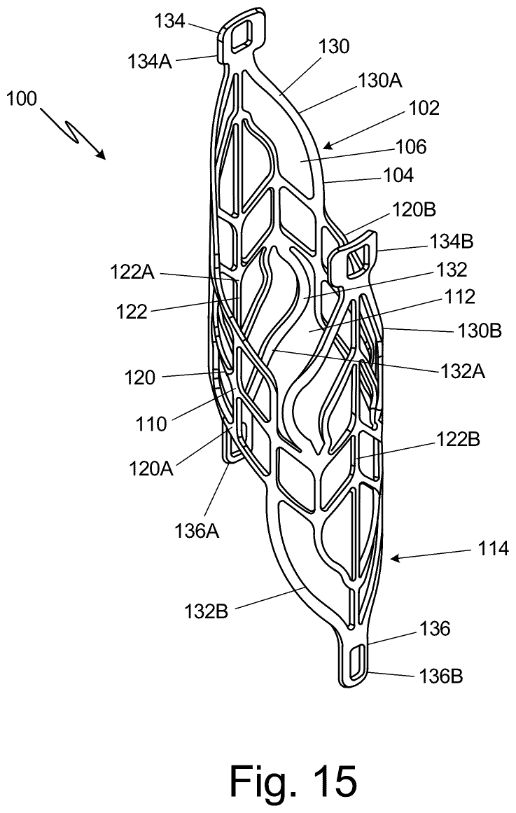

FIG. 14A is a perspective view of shunt device 100. FIG. 14B is a side view of shunt device 100. FIG. 14C is a bottom view of shunt device 100. FIG. 15 is a perspective view of shunt device 100 in a collapsed configuration. FIGS. 14A, 14B, 14C and 15 will be discussed together. Shunt device 100 includes body 102, which is formed of struts 104 and openings 106. Body 102 includes central flow tube 110, flow path 112, and arms 114. Central flow tube 110 has side walls 120 (including side wall 120A and side wall 120B) and end walls 122 (including end wall 122A and end wall 122B). Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 14B further shows horizontal reference plane HP, end wall axis EA, and angle α. FIG. 14C further shows vertical reference plane VP.

Shunt device 100 is shown in an expanded configuration in FIGS. 14A-14C. Shunt device 100 is formed of a super-elastic material that is capable of being compressed into a catheter for delivery into the body. Shunt device 100 is shown in a compressed configuration in FIG. 15. Upon delivery into the body, shunt device 100 will expand back to its relaxed, or expanded, shape. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded. Shunt device 100 can be sterilized before being delivered into the body.

Body 102 includes central flow tube 110 that forms a center portion of shunt device 100. Central flow tube 110 is tubular in cross-section, but is formed of struts 104 and openings 106. Central flow tube 110 can be positioned in a puncture in a tissue wall and holds the tissue wall open. Flow path 112 is an opening extending through central flow tube 110. Flow path 112 is the path through which blood flows through shunt device 100. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus, central flow tube 110 holds the tissue wall open so blood can flow from the left atrium to the coronary sinus through flow path 112. Struts 104 of central flow tube 110 form a cage of sorts that is sufficient to hold the tissue wall open around central flow tube 110. Central flow tube 110 is designed to have a thickness that approximates the thickness of the tissue wall between the left atrium and the coronary sinus.

Central flow tube 110 has side walls 120 and end walls 122. Side wall 120A and side wall 120B form opposing sides of central flow tube 110. End wall 122A and end wall 122B form opposing ends of central flow tube 110. End wall 122A and end wall 122B each extend between and connect to side wall 120A and side wall 120B to form a circular opening that defines flow path 112. Struts 104 of central flow tube 110 define generally parallelogram-shaped openings 106 in central flow tube 110. Struts 104 of side walls 120 form an array of parallelogram-shaped openings 106 in side walls 120. Side walls 120 and end walls 122 form a tubular lattice for central flow tube 110.

As shown in FIG. 14B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted. End walls 122 are angled with respect to horizontal reference plane HP. As shown in FIG. 14B, end walls 122 extend along end wall axis EA that extends at angle α with respect to horizontal reference plane HP. Angle α can be between 15° and 90°. Alternatively, angle α can be between 30° and 75°. Alternatively, angle α can be between 60° and 65°.

Arms 114 of shunt device 100 include two distal arms 130 and two proximal arms 132. Arms 114 extend outward from end walls 122 of central flow tube 110 when shunt device 100 is in an expanded configuration. Distal arm 130A is connected to and extends away from end wall 122A, and distal arm 130B is connected to and extends away from end wall 122B. Proximal arm 132A is connected to and extends away from end wall 122A, and proximal arm 132B is connected to and extends away from end wall 122B. When shunt device 100 is implanted in the tissue wall between the left atrium and the coronary sinus, distal arms 130 will be positioned in the left atrium and proximal arms 132 will be positioned in the coronary sinus.

Distal arms 130 and proximal arms 132 curl outward from end walls 122. As shown in FIG. 14C, distal arm 130A and distal arm 130B extend outwards from central flow tube 110 in opposite directions parallel to vertical reference plane VP. Distal arm 130A has a longer length than distal arm 130B. Proximal arm 132A and proximal arm 132B extend outwards from central flow tube 110 in opposite directions parallel to vertical reference plane VP. Proximal arm 132A has a shorter length than proximal arm 132B. Distal arm 130A has generally the same length and shape as proximal arm 132B, and distal arm 130B has generally the same length and shape as proximal arm 132A. As such, shunt device 100 is inversely symmetrical across horizontal reference plane HP, as shown in FIG. 14B.

Shunt device 100 is generally elongated longitudinally but is relatively narrow laterally. Stated another way, distal arms 130 and proximal arms 132 are not annular or circular, but rather extend outward generally in only one plane. As shown in FIG. 14B, shunt device 100 has a generally H-shape when viewing a side of shunt device 100. The elongated shape of shunt device 100 means that when compressed it elongates along a line, as shown in FIG. 15, so as to better fit within a catheter.

Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B. Terminal ends 134 of distal arms 130 and terminal ends 136 of proximal arms 132 converge towards one another. Distal arms 130 and proximal arms 132 form two pairs of arms. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. The gap between terminal ends 134 and terminal ends 136 is sized to be slightly smaller than an approximate thickness of the tissue wall between the left atrium and the coronary sinus. This allows distal arms 130 and proximal arms 132 to flex outwards and grip the tissue wall when implanted to help hold shunt device 100 in place in the tissue wall. Terminal ends 134 of distal arms 130 and terminal ends 136 of proximal arms 132 can also have openings that are configured to engage a delivery tool to facilitate implantation of shunt device 100, for example actuating rods of a delivery tool.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the intra-cardiac flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the blood flow through the coronary sinus.

Shunt device 100 and other examples of shunt devices are described in further detail in U.S. Pat. No. 9,789,294, filed on Oct. 6, 2016, issued on Oct. 17, 2017, and entitled “Expandable Cardiac Shunt,” the disclosure of which is incorporated by reference in its entirety. Shunt device 100 can be implanted in a tissue wall using a catheter-based method know in the art, for example as described in U.S. Pat. No. 9,789,294.

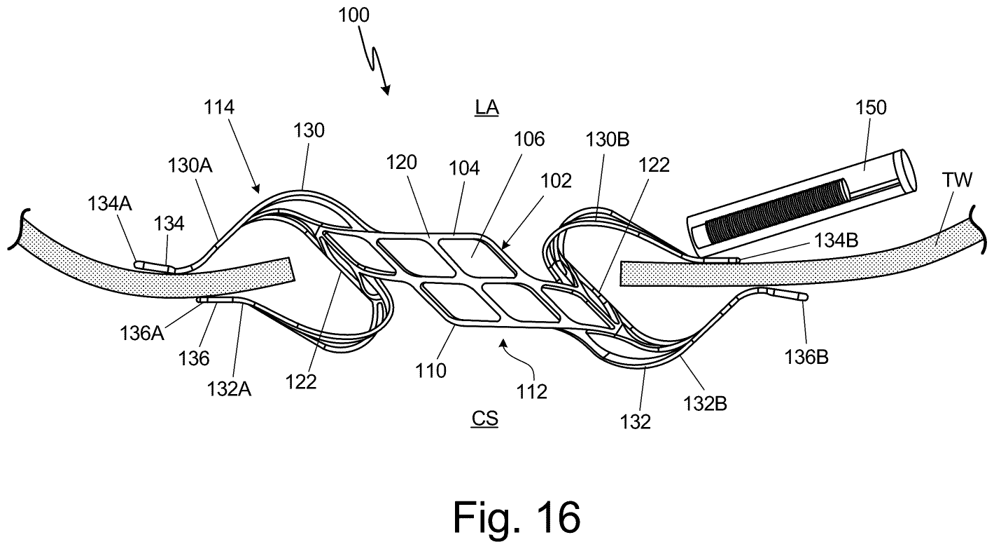

FIG. 16 is a side view of shunt device 100 with sensor 150 and anchored to tissue wall TW. Shunt device 100 includes body 102, which is formed of struts 104 and openings 106. Body 102 includes central flow tube 110, flow path 112, and arms 114. Central flow tube 110 has side walls 120 and end walls 122. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 16 further shows sensor 150, tissue wall TW, left atrium LA, and coronary sinus CS.

Shunt device 100 is described above in reference to FIGS. 14A-15. Shunt device 100 as shown in FIG. 16 further includes sensor 150. Shunt device 100 is shown implanted in tissue wall TW. In the example shown in FIG. 16, sensor 150 will be positioned in left atrium LA when shunt device 100 is implanted in tissue wall TW. Sensor 150 is attached to distal arm 130B of shunt device 100 in the example shown in FIG. 16, but can be attached to distal arm 130A in alternate examples. In further examples, sensor 150 can be attached to proximal arm 132A or proximal arm 132B to be positioned in coronary sinus CS. Alternatively, an additional sensor can be included on shunt device 100 to position a sensor in both left atrium LA and coronary sinus CS. Sensor 150 can be integrally formed with shunt device 100 or attached to shunt device 100 using any suitable mechanism.

Sensor 150 can be a pressure sensor to sense a pressure in the left atrium. In other examples, sensor 150 can be any sensor to measure a parameter in the left atrium. Sensor 150 can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150 can include alternate or additional components in other examples. Further, the components of sensor 150 can be held in a sensor housing that is hermetically sealed.

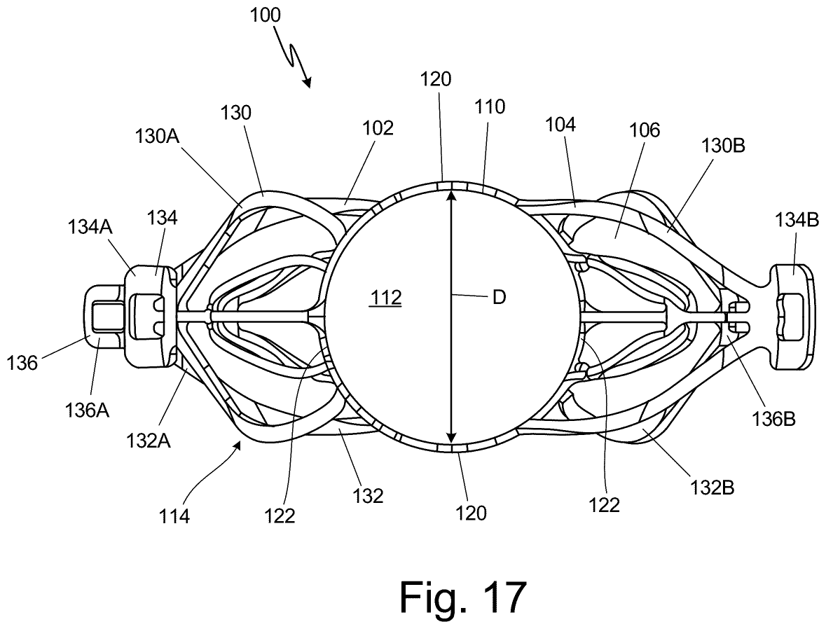

FIG. 17 is a top view looking through angled central flow tube 110 of shunt device 100. Shunt device 100 includes body 102, which is formed of struts 104 and openings 106. Body 102 includes central flow tube 110, flow path 112, and arms 114. Central flow tube 110 has side walls 120 and end walls 122. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 17 further shown diameter D.

Shunt device 100 is described above in reference to FIGS. 14A-15. Shunt device 100 can optionally include a sensor, as shown in FIG. 16. Central flow tube 110 of shunt device 100 defines a generally circular opening when viewed along the angled axis of central flow tube 110, as shown in FIG. 17. Central flow tube 110 has diameter D between 0.04 inches (1 millimeters) and 0.47 inches (12 millimeters). Alternatively, central flow tube 110 has diameter D of 0.12 inches (3 millimeters) and 0.39 inches (10 millimeters). Alternatively, central flow tube 110 has diameter D of 0.20 inches (5 millimeters) and 0.32 inches (8 millimeters). Alternatively, central flow tube 110 has diameter D of 0.28 inches (7 millimeters).

Diameter D of central flow tube 110 is selected to maintain a pulmonary to systemic flow ratio between 1.2 and 1.4, and preferably around 1.2. The pulmonary to systemic flow ratio is a ratio of the flow through the pulmonary system (i.e., flow through the lungs) to the flow through the systemic system (i.e., flow through the rest of the body). In healthy patients without a shunt device or opening between the right heart and the left heart, the pulmonary to systemic flow ratio is around 1. This allows the pulmonary flow and systemic flow to flow at equal rates to prevent one side from filling up over the other.

Shunt device 100 has an impact on the pulmonary to systemic flow ratio, as shunt device 100 moves blood from the left heart to the right heart. Diameter D of shunt device 100 is selected to maintain the pulmonary to systemic flow ratio between 1.2 and 1.4, and preferably around 1.2. The body can adjust the mismatch of flow when the change to the pulmonary to systemic flow ratio is small. However, if the change to the pulmonary to systemic flow ratio is too great, a patient can develop heart failure due to the mismatch of flows between the pulmonary system and the systemic system. A pulmonary to systemic flow ratio between 1.2 and 1.4 is low enough to allow the body to adjust to the mismatch in flow between the pulmonary system and the systemic system while also decreasing a pressure in the left atrium by shunting blood to the right atrium. Further, this prevents the right atrium from being volume overloaded. A pulmonary to systemic flow ratio between 1.2 and 1.4 can be achieved when diameter D of shunt device 100 is between 0.20 inches (5 millimeters) and 0.32 inches (8 millimeters).

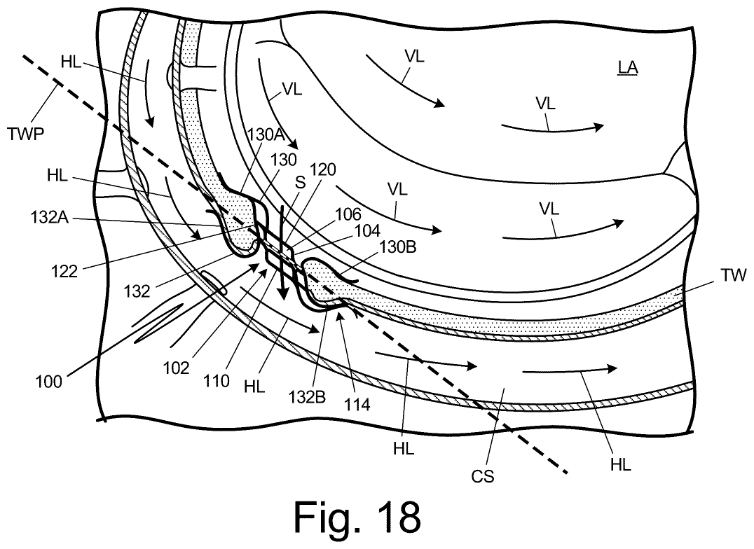

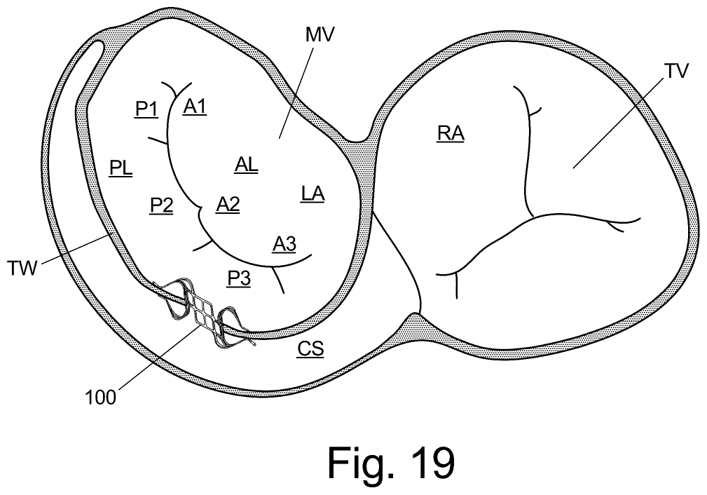

FIG. 18 is a cross-sectional view through heart H showing shunt device 100 implanted in tissue wall TW between left atrium LA and coronary sinus CS of heart H. FIG. 19 is a cross-sectional view through heart H showing shunt device 100 implanted in tissue wall TW and a flow path from left atrium LA to right atrium AT through shunt device 100. Shunt device 100 includes body 102, which is formed of struts 104 and openings 106. Body 102 includes central flow tube 110, flow path 112, and arms 114. Central flow tube 110 has side walls 120 and end walls 122. Arms 114 include distal arms 130 (including distal arm 130A and distal arm 130B) and proximal arms 132 (including proximal arm 132A and proximal arm 132B). FIGS. 18-19 also shows tissue wall TW, left atrium LA and coronary sinus CS. FIG. 18 further shows arrows VL, arrows HL, arrow S, and dashed line TWP. FIG. 19 further shows right atrium RA, tricuspid valve TV, and mitral valve MV, which includes anterior leaflet AL and posterior leaflet PL. Anterior leaflet AL has region A1, region A2, and region A3. Posterior leaflet PL has region P1, region P2, and region P3.

Shunt device 100 is described above in reference to FIGS. 14A, 14B, 14C, and 15. Shunt device 100 can optionally include a sensor, as shown in FIG. 16. Shunt device 100 is positioned in tissue wall TW between left atrium LA and coronary sinus CS. FIG. 18 includes arrows VL that represent the left-sided flow vortex in left atrium LA, arrows HL that represent the helical flow pattern in coronary sinus CS, and arrow S that represents the flow of blood from left atrium LA to coronary sinus CS.

Shunt device 100 is positioned in tissue wall TW so that distal arms 130 and proximal arms 132 are positioned on opposite sides of tissue wall TW, respectively. Tissue wall TW includes the inner and outer wall of the left atrial wall, the inner and outer wall of the coronary sinus wall, and any tissue that may be between the left atrial wall and the coronary sinus wall. Distal arms 130 and proximal arms 132 compress tissue wall TW and hold the layers of tissue wall TW together to form a water tight seal around shunt device 100. Further, distal arms 130 and proximal arms 132 anchor shunt device 100 to tissue wall TW.

Shunt device 100 has central flow tube 110 that is angled with respect to tissue wall TW immediately adjacent to shunt device 100. As shown in FIG. 18, tissue wall TW is not a straight wall. However, end walls 122 of central flow tube 110 will form an angle with respect to a plane that extends through tissue wall TW immediately adjacent to shunt device 100, represented by dashed line TWP in FIG. 18. End walls 122 of central flow tube 110 will be angled with respect to tissue wall TW between 150 and 90°. Alternatively, end walls 122 of central flow tube 110 will be angled with respect to tissue wall TW between 300 and 75°. Alternatively, end walls 122 of central flow tube 110 will be angled with respect to tissue wall TW between 600 and 65°.

As shown in FIG. 18, shunt device 100 is positioned in tissue wall TW so that the blood from the left-sided flow vortex (shown with arrows V) can flow smoothly through flow path 112 of central flow tube 110 of shunt device 100. The blood flows along the left-sided flow vortex in left atrium LA down towards the mitral valve. Shunt device 100 is positioned in tissue wall TW so that the left-sided flow vortex of the blood in left atrium LA is guided through shunt device 100. Further, shunt device 100 is positioned at an angle with respect to tissue wall TW to allow the left-sided flow vortex in left atrium LA to flow smoothly into flow path 112 of central flow tube 110 of shunt device 100. This provides minimal disruption to the left-sided flow vortex in left atrium LA, while still shunting blood from left atrium LA into coronary sinus CS to relieve the pressure buildup in the left heart.

Further, positioning shunt device 100 in tissue wall TW at an angle allows the blood to flow through flow path 112 of central flow tube 110 of shunt device 100 at an angle to join smoothly into the helical flow pattern (shown with arrows H) of blood in coronary sinus CS. This provides minimal disruption to the natural helical flow pattern in coronary sinus CS and prevents turbulent flow in coronary sinus CS. If the flow coming through shunt device 100 disrupts the flow in coronary sinus CS and creates turbulence in coronary sinus CS, it can jet into the right atrium and disrupt the right-sided flow vortex in the right atrium. If the flow of blood moving through flow path 112 of shunt device 100 smoothly joins into the helical flow pattern of blood in coronary sinus CS, there will be minimal to no disruption in coronary sinus CS and the blood flowing into the right atrium from coronary sinus CS will not be disrupted. Further, it is hypothesized that the increased flow of blood exiting coronary sinus CS into the right atrium can smoothly join and enhance the right-sided flow vortex formed in the right atrium, particularly for patients who are experiencing a disruption of the right-sided flow vortex in the right atrium due to age or disease.

Coronary sinus CS extends along a floor of left atrium LA adjacent to mitral valve MV. Shunt device 100 can be positioned adjacent to mitral valve MV to minimize the disturbance to the intra-cardiac flow patterns in left atrium LA and coronary sinus CS. Ideally, shunt device 100 is positioned to enhance or restore the right-sided flow vortex in right atrium RA. As shown in FIG. 19, shunt device 100 can be positioned in tissue wall TW adjacent to posterior leaflet PL of mitral valve MV. Specifically, shunt device 100 can be positioned in tissue wall TW adjacent to a mid-portion of posterior leaflet PL of mitral valve MV. More specifically, shunt device 100 can be positioned in tissue wall TW adjacent to an area between region P1 and region P3 of posterior leaflet PL of mitral valve MV. More specifically, shunt device 100 can be positioned in tissue wall TW adjacent to region P2 and region P3 of posterior leaflet PL of mitral valve MV.

Shunt device 100 can be positioned in coronary sinus CS between 0.59 inches (15 millimeters) and 1.18 inches (30 millimeters) from an ostium of coronary sinus CS. Alternatively, shunt device 100 can be positioned in coronary sinus CS between 0.59 inches (15 millimeters) and 0.98 inches (25 millimeters) from the ostium of coronary sinus CS. Alternatively, shunt device 100 can be positioned in coronary sinus CS between 0.59 inches (15 millimeters) and 0.79 inches (20 millimeters) from the ostium of coronary sinus CS. Alternatively, shunt device 100 can be positioned in coronary sinus CS 0.79 inches (20 millimeters) from the ostium of coronary sinus CS.

The position of shunt device 100 in coronary sinus CS is selected to provide sufficient space for shunt device 100 to be implanted in tissue wall TW. Further, the position of shunt device 100 in coronary sinus CS is selected to prevent mitral regurgitation. Additionally, the position of shunt device 100 is coronary sinus CS is selected to allow the blood flowing from left atrium LA into coronary sinus CS to fully join with the helical flow pattern of blood in coronary sinus CS before entering into right atrium RA. Shunt device 100 should be positioned to preserve a length of coronary sinus CS downstream of shunt device 100 in which the flow of blood from left atrium LA can fully join into the helical flow pattern of blood in coronary sinus CS before entering right atrium RA.

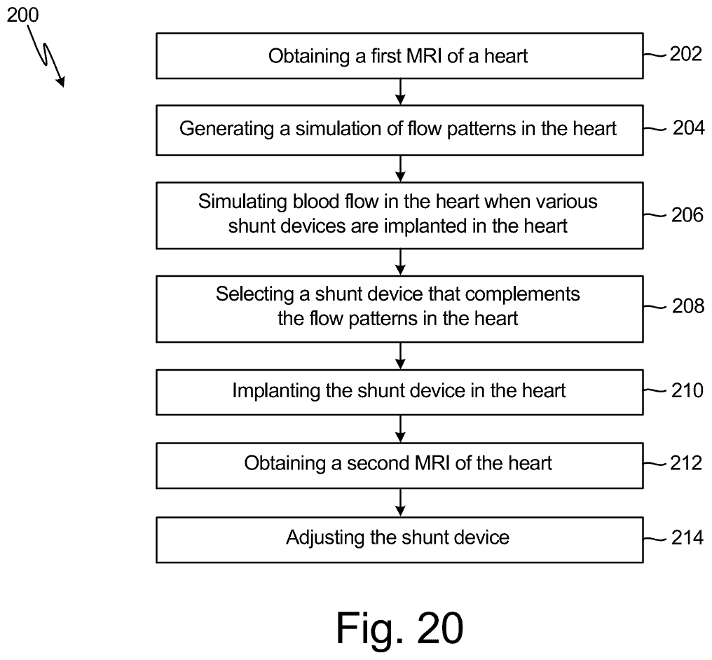

FIG. 20 is a flowchart showing method 200 for selecting a shunt device for implantation in the heart. Method 200 includes steps 202-214. A shunt device, or alternatively a deviceless shunt, is selected according to method 200 to minimize or eliminate disruption of or enhance flow patterns of blood flow in a heart of a patient.

Step 202 includes obtaining a first MRI of a heart. The first MRI (magnetic resonance imaging) can visualize the flow patterns of blood flow in the heart of the patient. Specifically, the first MRI can visualize the flow patterns of blood flow in the right atrium, the left atrium, and/or the coronary sinus of the heart of the patient.

The first MRI can be a 4D MRI that visualizes the flow patterns of blood flow in the heart of a patient. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

Step 204 includes generating a simulation of the flow patterns in the heart. The simulation of the flow patterns in the heart can be generated based on the first MRI. This allows the simulation to be patient specific. The simulation will simulate the flow patterns in the heart of the patient, and specifically in the right atrium, the left atrium, and the coronary sinus of the heart. The simulation can also simulate the volumes, sizes, geometries, compliances, and blood pressures of the chambers of the heart based on data from the first MRI. The simulation can be generated using any suitable software program.

Step 206 includes simulating blood flow in the heart when various shunt devices are implanted in the heart. The simulated blood flow in the heart is modulated by the shunt devices to simulate the impact of the shunt devices on the flow patterns in the heart. The blood flow in the heart can be simulated when the heart includes shunt devices, for example, having varying cross-sectional areas (e.g., varying diameters) of a flow path of the shunt device, varying angles of central flow tubes of the shunt devices with respect to the tissue wall in which the shunt devices are implanted, and/or varying placement of the shunt devices along the coronary sinus.

Step 208 includes selecting the shunt device that complements the flow patterns in the heart. The shunt device is selected to minimize or eliminate disruption of flow patterns in the heart. Specifically, step 208 can include selecting a design of the shunt device and a placement of the shunt device along the coronary sinus that complements the flow patterns in the heart. More specifically, a cross-sectional area of a flow path of the shunt device can be selected to complement the flow patterns in the heart; an angle of a central flow tube of the shunt device with respect to the tissue wall in which the shunt device is implanted can be selected to complement the flow pattern in the heart; and placement of the shunt device along the coronary sinus can be selected to complement the flow pattern in the heart.

The right atrium of the heart has a right-sided flow vortex as a natural flow pattern in the heart. The design of the shunt device and the placement of the shunt device along the coronary sinus can be selected to complement the right-sided flow vortex in the right atrium of the heart. The left atrium of the heart has a left-sided flow vortex as a natural flow pattern in the heart. The design of the shunt device and the placement of the shunt device along the coronary sinus can be selected to complement the left-sided flow vortex in the left atrium of the heart. The coronary sinus has a helical flow pattern. The design of the shunt device and the placement of the shunt device along the coronary sinus can be selected to complement the helical flow pattern in the coronary sinus.

In an alternate example, step 208 can include selecting a design of a shunt device and a placement of a shunt device along the coronary sinus that enhances the flow pattern in the right atrium of the heart and/or reestablishes the natural flow pattern in the right atrium of the heart. Specifically, if the patient has lost the right-sided flow vortex of blood flow in the right atrium of the heart due to age, disease, or anatomical defects, the design of the shunt device and the placement of the shunt device along the coronary sinus can be selected to reestablish the right-sided flow vortex of blood flow in the right atrium of the heart.

Step 210 includes implanting the shunt device in the heart. The shunt device can be implanted using any suitable method. For example, the shunt device can be implanted according to methods described in U.S. Pat. No. 9,789,294, filed on Oct. 6, 2016, issued on Oct. 17, 2017, and entitled “Expandable Cardiac Shunt,” the disclosure of which is incorporated by reference in its entirety.

Step 212 includes obtaining a second MRI of the heart. The second MRI (magnetic resonance imaging) can visualize the flow patterns of blood flow in the heart of the patient after the shunt device has been implanted. Specifically, the second MRI can visualize the flow patterns of blood flow in the right atrium, the left atrium, and/or the coronary sinus of the heart of the patient after the shunt device has been implanted.

The second MRI can be a 4D MRI that visualizes the flow patterns of blood flow in the heart of a patient after the shunt device has been implanted. Additionally, the 4D MRI can measure volumes in the chambers of the heart, sizes of the chambers of the heart, geometries of the chambers of the heart, compliances of the chambers of the heart, and/or blood pressures in the chambers of the heart. The 4D MRI can also track movement of the chambers of the heart and movement of the tricuspid valve (also known as tricuspid annular plan systolic excursion (TAPSE)).

The second MRI is obtained to confirm that the shunt device complements (e.g., has minimal to no disruption of) the flow patterns in the heart. Further, the second MRI can be obtained to determine whether the shunt device has enhanced and/or reestablished the natural flow patterns in the heart. Specifically, the second MRI can be obtained to determine whether the shunt device has reestablished a right-sided flow vortex in a right atrium of the heart.

The second MRI can also confirm the overall health of the heart after the shunt device has been implanted. Specifically, the volumes in the chambers of the heart, sizes of the chambers of the heart, geometries of the chambers of the heart, compliances of the chambers of the heart, and/or blood pressures in the chambers of the heart from the second MRI can be compared to the same readings from the first MRI to confirm overall health of the heart. In one example, the volumes, sizes, geometries, and/or compliances of the chambers of the heart can be analyzed to determine if the left side of the heart has experiences remodeling (shrinkage) due to the reduced blood pressure on the left side of the heart after the shunt device has been implanted. Further, the volumes, sizes, geometries, and/or compliances of the chambers of the heart can be analyzed to determine if the right side of the heart is being overloaded due the increased blood pressure in the right side of the heart.

Step 214 includes adjusting the shunt device. The shunt device can be adjusted if the second MRI shows that the implantation of the shunt device has not had the desired effect on the flow patterns in or overall health of the heart. Specifically, a cross-sectional area of a flow path of the shunt device can be adjusted. For example, a central flow tube of the shunt device can be made of an adjustable stent-like structure that can made wider or narrower. Further, a component can be added to a flow path of a shunt device to reduce a cross-sectional area of the flow path of the shunt device. Alternatively, the shunt device can be replaced with a shunt device having a different design.

Method 200 as described herewith can be used to aid in the selection and implantation of any suitable shunt device. In one example, method 200 can be used to aid in the selection and implantation of shunt device 100 (shown in FIGS. 14A-19). Various shunt devices 100 can be provided that have varying diameters of flow paths 112 and varying angles of central flow tubes 110 with respect to the tissue wall in which shunt devices 100 are implanted. Method 200 can be used to select a design of shunt device 100 and a placement of shunt device 100 along the coronary sinus that will complement (e.g., minimize or eliminate disruptions) the flow patterns in the heart.

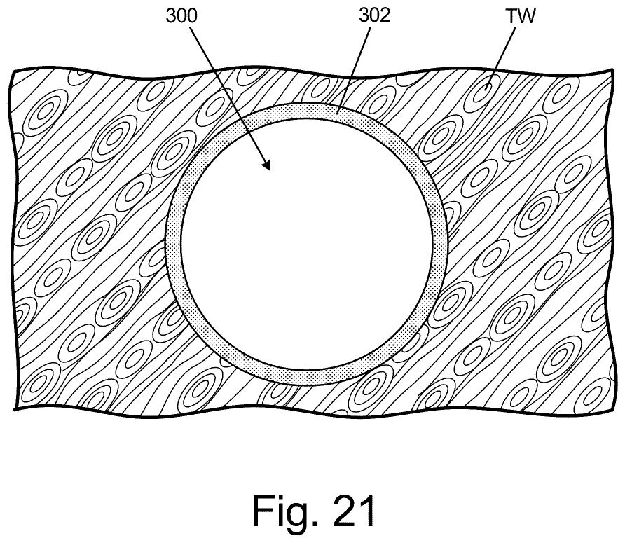

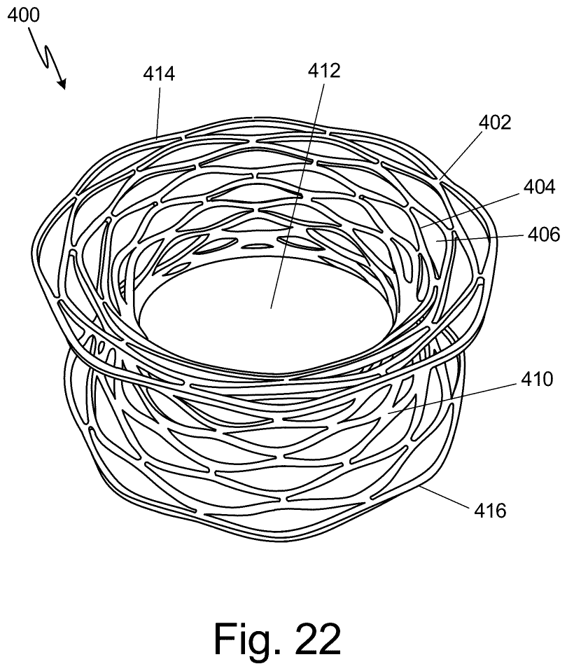

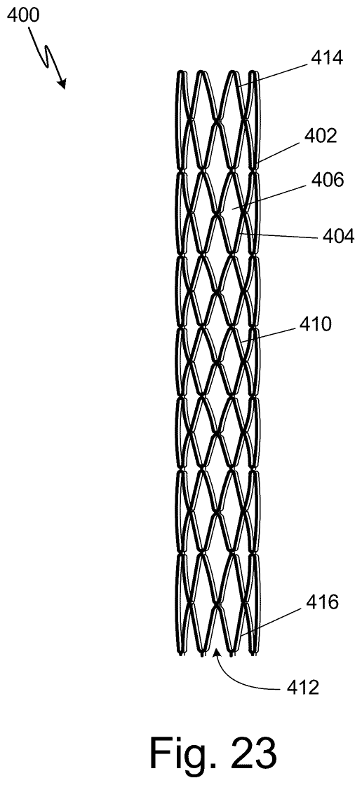

In alternate examples, method 200 can be used with any other design of a shunt device. Method 200 can also be used to select a size and placement of a deviceless shunt in the tissue wall between the coronary sinus and left atrium of the heart. Alternate examples of shunts and shunt devices are described below with reference to FIGS. 21-23. A deviceless shunt is shown in FIG. 21. An armless shunt device is shown in FIGS. 22-23.

FIG. 21 is a schematic view of shunt 300 in tissue wall TW between a coronary sinus and a left atrium. FIG. 21 shows shunt 300, areas of tissue 302, and tissue wall TW.

Shunt 300 is formed in tissue wall TW between a coronary sinus and a left atrium. Shunt 300 can be formed in tissue wall TW using any suitable method. For example, shunt 300 can be punctured into, ablated through, burned through, cut out of, removed from, or cauterized through tissue wall TW. Shunt 300 is a deviceless shunt in tissue wall TW.

One or more areas of tissue 302 around and/or near shunt 300 can also be treated to prevent, inhibit, reduce, and/or contain tissue growth in an area around shunt 300. The method of treating area of tissue 302 may involve ablating, burning, cutting, removing, cauterizing, scarring, and/or otherwise treating the one or more areas of tissue 302. Area of tissue 302 may comprise a portion of an outer surface of tissue wall TW (e.g., on a left atrium side or coronary sinus side of tissue wall TW) and/or on an inner surface of tissue wall TW (e.g., within the opening of shunt 300 in tissue wall TW).

Various tools may be delivered for use in treating the one or more areas of tissue 302. For example, a laser or similar device may be used to remove and/or burn area of tissue 302. Treatment of the one or more areas of tissue 302 may involve electrical ablation and/or use of an electrical cauterizing tool to cause a controlled scarring pattern and/or block electrical transmission at area of tissue 302.

As shown in FIG. 21, area of tissue 302 may have an elliptical (e.g., circular) shape and/or may approximate a shape of shunt 300 in tissue wall TW. However, the one or more areas of tissue 302 may have any size and/or shape. For example, area of tissue 302 may not comprise a complete ellipse and may instead comprise a linear, jagged, curved, non-linear, etc. shape. In some embodiments, multiple areas of tissue 302 may be created around shunt 300. For example, multiple elliptical or semi-elliptical areas of tissue 302 having different sizes and/or radii may form an elliptical or other shape. The one or more areas of tissue 302 may represent multiple levels of treated tissue along a lateral axis extending from shunt 300. A first area of tissue 302 may be positioned distal to shunt 300 and a second area of tissue 302 may be positioned proximal to shunt 300.

Method 200 described above in reference to FIG. 20 can be used to select and form shunt 300 in tissue wall TW, with slight modifications. A first MRI of the heart will be obtained, and a simulation of flow patterns in the heart will be generated. Blood flow in the heart will be simulated for various shunts having varying diameters and placements along the coronary sinus. A diameter of shunt 300 and a placement of shunt 300 along the coronary sinus will be selected that complements the flow patterns in the heart. Shunt 300 will then be created in the tissue wall of the heart using any suitable method. A second MRI of the heart will be obtained. Shunt 300 can be made larger, if needed, to adjust the impact of shunt 300 on the flow patterns in the heart.

FIG. 22 is a perspective view of armless shunt device 400. FIG. 23 is a side view of armless shunt device 400. Armless shunt device 400 includes body 402, which is formed of struts 404 and openings 406. Body 402 includes central flow tube 410, flow path 412, distal flange 414, and proximal flange 416.

Shunt device 400 is shown in an expanded configuration in FIG. 22. Shunt device 400 is formed of a super-elastic material that is capable of being compressed into a catheter for delivery into the body. Shunt device 400 is shown in a compressed configuration in FIG. 23. Upon delivery into the body, shunt device 400 will expand back to its relaxed, or expanded, shape. Shunt device 400 has body 402 that is formed of interconnected struts 404. Openings 406 in body 402 are defined by struts 404. Body 402 of shunt device 400 is formed of struts 404 to increase the flexibility of shunt device 400 to enable it to be compressed and expanded. Shunt device 400 can be sterilized before being delivered into the body.

Body 402 includes central flow tube 410 that forms a center portion of shunt device 400. Central flow tube 410 is tubular in cross-section, but is formed of struts 404 and openings 406. Central flow tube 410 can be positioned in a puncture in a tissue wall and holds the tissue wall open. Flow path 412 is an opening extending through central flow tube 410. Flow path 412 is the path through which blood flows through shunt device 400. Distal flange 414 is formed at a distal end of body 402, and proximal flange 416 is formed at a proximal end of body 402. Distal flange 414 and proximal flange 416 extend radially outward from central flow tube 410. Distal flange 414 and proximal flange 416 are formed of struts 404 and openings 406.

Openings 406 vary in size. As shown in FIG. 23, openings 406 between struts 404 are smaller in a center of body 402 and get larger in distal and proximal directions. More specifically, openings 405 are smaller in central flow tube 410 and larger in distal flange 414 and proximal flange 416. Openings 406 in distal flange 414 and proximal flange 416 are smaller adjacent central flow tube 410 and are larger adjacent distal end and proximal end, respectively. Openings 406 being smallest in central flow tube 410 means that central flow tube 410 will have the smallest diameter when shunt device 400 is in an expanded position. Openings 406 varying in size in a distal and proximal direction towards distal flange 414 and proximal flange 416, respectively, means that distal flange 414 and proximal flange 416 will taper from a smaller diameter to a larger diameter when shunt device 400 is in an expanded position. Struts 404 have straight regions and triangular peaks which form generally diamond shaped openings 406 between struts 404. As a result of this design, the angle of each peak increases as shunt device 400 is expanded, which causes longitudinal foreshortening to shorten the length of shunt device 400.

When shunt device 400 is implanted in the tissue wall between the left atrium and the coronary sinus, central flow tube 410 holds the tissue wall open so blood can flow from the left atrium to the coronary sinus through flow path 412. Struts 404 of central flow tube 410 form a cage of sorts that is sufficient to hold the tissue wall open around central flow tube 410. Central flow tube 410 is designed to have a thickness that approximates the thickness of the tissue wall between the left atrium and the coronary sinus.

When shunt device 400 is implanted in the tissue wall between the left atrium and the coronary sinus, distal flange 414 will be positioned in the left atrium and proximal flange 416 will be positioned in the coronary sinus. When implanted in the tissue wall, distal flange 414 and proximal flange 416 are designed such that the projection of distal flange 414 and proximal flange 416 into the left atrium and the coronary sinus, respectively, is minimized. This minimizes the disruption of the intra-cardiac flow patterns in the left atrium and the coronary sinus.

Shunt device 400 can be implanted in a tissue wall using a catheter-based method know in the art, for example as described in U.S. Publication No. 2020/0254228, filed on Feb. 7, 2020, published on Aug. 13, 2020, and entitled “Rivet Shunt and Method of Deployment,” the disclosure of which is incorporated by reference in its entirety.

Method 200 described above in reference to FIG. 20 can be used to select one shunt device 400 for implantation into a tissue wall. Shunt device 400 can have numerous designs to, for example, vary a cross-sectional area of flow path 412 of shunt device 400. Shunt device 400 is one example of an armless shunt device that can be used in connection with method 200. Any suitable armless shunt device can be used in other examples.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible embodiments of the present invention.

A method of selecting a shunt device for implantation in a heart includes obtaining a first MRI of the heart, and generating a simulation of flow patterns of blood flow in the heart. Blood flow in the heart is simulated when various shunt devices are implanted in the heart. The shunt device that complements the flow patterns of blood flow in the heart is selected, and the shunt device is implanted in the heart.

The method of the preceding paragraph can optionally include, additionally and/or alternatively, any one or more of the following features, configurations and/or additional components:

Wherein obtaining the first MRI of the heart further includes obtaining a 4D MRI of the heart to visualize the flow patterns of blood flow in the heart.

Wherein obtaining the first MRI of the heart further includes obtaining a 4D MRI of the heart to visualize the flow patterns of blood flow in a right atrium, a left atrium, and/or a coronary sinus of the heart.

Wherein generating the simulation of the flow patterns of blood flow in the heart further includes generating a simulation of the flow patterns of blood flow in a right atrium, a left atrium, and/or a coronary sinus of the heart.

Wherein simulating blood flow in the heart when the various shunt devices are implanted in the heart further includes simulating blood flow in the heart when the shunt devices having varying cross-sectional areas of a flow path of the shunt device, varying angles of central flow tubes of the shunt devices with respect to a tissue wall in which the shunt devices are implanted, and/or varying placement of the shunt devices along the coronary sinus are implanted in the heart.

Wherein selecting the shunt device that complements the flow patterns of blood flow in the heart further includes selecting a design of the shunt device that complements the flow patterns of blood flow in the heart.

Wherein selecting the design of the shunt device that complements the flow patterns of blood flow in the heart further includes selecting a cross-sectional area of a flow path of the shunt device that complements the flow patterns of blood flow in the heart.

Wherein selecting the design of the shunt device that complements the flow patterns of blood flow in the heart further includes selecting an angle of a central flow tube of the shunt device with respect to the tissue wall in which the shunt device is implant that complements the flow patterns of blood flow in the heart.

Wherein selecting the shunt device that complements the flow patterns of blood flow in the heart further includes selecting the design of the shunt device that complements a right-sided flow vortex in a right atrium of the heart.

Wherein selecting the shunt device that complements the flow patterns of blood flow in the heart further includes selecting a placement of the shunt device along the coronary sinus that complements the flow patterns of blood flow in the heart.

Wherein selecting the placement of the shunt device along the coronary sinus that complements the flow patterns of blood flow in the heart further includes selecting a placement of the shunt device along the coronary sinus that complements a right-sided flow vortex in a right atrium of the heart.

Wherein selecting the shunt device that complements the flow patterns of blood flow in the heart further includes selecting the shunt device that complements a right-sided flow vortex in a right atrium of the heart, a left-sided flow vortex in a left atrium of the heart, and/or a helical flow pattern of blood flow in a coronary sinus of the heart.

Wherein selecting the shunt device that complements the flow patterns of blood flow in the heart further includes selecting a design of the shunt device and a placement of the shunt device along the coronary sinus that enhances a right-sided flow vortex of blood flow in a right atrium of the heart.

Wherein selecting the shunt device that complements the flow patterns of blood flow in the heart further includes selecting a design of the shunt device and a placement of the shunt device along the coronary sinus that reestablishes a right-sided flow vortex of blood flow in a right atrium of the heart.

The method further includes obtaining a second MRI of the heart.

Wherein obtaining the second MRI of the heart further includes obtaining a 4D MRI of the heart to visualize the flow patterns of blood flow in the heart.

Wherein obtaining the 4D MRI of the heart to visualize the flow patterns of blood flow in the heart further includes obtaining the 4D MRI of the heart to visualize the flow patterns of blood flow in a right atrium, a left atrium, and/or a coronary sinus of the heart.

Wherein obtaining the first MRI of the heart further includes measuring a volume of one or more chambers of the heart; measuring a size of one or more chamber of the heart; measuring a geometry of one or more chambers of the heart; measuring a compliance of one or more chambers of the heart; measuring a blood pressure in one or more chambers of the heart; tracking a movement of one or more chambers of the heart; and/or tracking a movement of a tricuspid valve of the heart, and wherein obtaining the second MRI of the heart further includes measuring a volume of one or more chambers of the heart; measuring a size of one or more chamber of the heart; measuring a geometry of one or more chambers of the heart; measuring a compliance of one or more chambers of the heart; measuring a blood pressure in one or more chambers of the heart; tracking a movement of one or more chambers of the heart; and/or tracking a movement of a tricuspid valve of the heart.

The method further includes comparing one or more of the volume, the size, the geometry, the compliance, the blood pressure, the movement of one or more chambers, and/or the movement of the tricuspid valve from the first MRI with one or more of the volume, the size, the geometry, the compliance, the blood pressure, the movement of one or more chambers, and/or the movement of the tricuspid valve from the second MRI to analyze an overall health of the heart.

The method further includes adjusting the shunt device.

The above method(s) can be performed on a living animal or on a simulation, such as on a cadaver, cadaver heart, anthropomorphic ghost, simulator (e.g., with body parts, heart, tissue, etc. being simulated).

A method of shunting blood between a left atrium and a right atrium of a heart includes positioning a shunt device in a tissue wall between the left atrium and a coronary sinus of the heart so that a flow path through a central flow tube of the shunt device is positioned to guide a flow of blood through the flow path of the shunt device to join with a natural flow pattern of blood flow within the coronary sinus. The blood is shunted from the left atrium to the coronary sinus through the flow path of the shunt device. The flow of blood through the shunt device is joined with the natural flow pattern of blood flow within the coronary sinus. The blood from the coronary sinus is moved into the right atrium via a natural orifice of the coronary sinus. The flow of blood from the coronary sinus is joined with a natural flow pattern of blood flow within the right atrium.

The method of the preceding paragraph can optionally include, additionally and/or alternatively, any one or more of the following features, configurations and/or additional components:

Wherein joining the flow of blood from the coronary sinus with the natural flow pattern of blood flow within the right atrium further includes joining the flow of blood from the coronary sinus with a right-sided flow vortex of blood flow within the right atrium.

The method further includes enhancing the right-sided flow vortex of blood flow within the right atrium.

The method further includes reestablishing the right-sided flow vortex of blood flow within the right atrium.

Wherein the blood moving from the coronary sinus into the right atrium includes a natural flow of blood from the coronary sinus and a shunted flow of blood from the left atrium into the coronary sinus through the flow path of the shunt device.

Wherein joining the flow of blood from the coronary sinus with the natural flow pattern of blood flow within the right atrium further includes joining the flow of blood from the coronary sinus with an inflow of blood from an inferior vena cava that flows into the natural flow pattern of blood flow within the right atrium.

Wherein joining the flow of blood through the shunt device with the natural flow pattern of blood flow within the coronary sinus further includes joining the flow of blood through the shunt device with a helical flow pattern of blood flow within the coronary sinus.

Wherein positioning the shunt device in the tissue wall between the left atrium and the coronary sinus further includes positioning the shunt device in the tissue wall between the left atrium and the coronary sinus of the heart so that the flow path through the central flow tube of the shunt device guides a natural flow pattern of blood flow within the left atrium through the central flow tube of the shunt device.

Wherein positioning the shunt device in the tissue wall between the left atrium and the coronary sinus further includes positioning the shunt device in the tissue wall between the left atrium and the coronary sinus of the heart so that the flow path through the central flow tube of the shunt device guides a left-sided flow vortex of blood flow within the left atrium through the central flow tube of the shunt device.

Wherein positioning the shunt device in the tissue wall between the left atrium and the coronary sinus of the heart further includes positioning the shunt device at an angle with respect to the tissue wall.

Wherein the angle is between 150 and 90°.

Wherein the angle is between 300 and 75°.

Wherein the angle is between 600 and 65°.

Wherein the central flow tube of the shunt device has a diameter between 0.04 inches (1 millimeters) and 0.47 inches (12 millimeters).

Wherein the central flow tube of the shunt device has a diameter between 0.12 inches (3 millimeters) and 0.39 inches (10 millimeters).

Wherein the central flow tube of the shunt device has a diameter between 0.1969 inches (5 millimeters) and 0.32 inches (8 millimeters).

Wherein the central flow tube of the shunt device has a diameter of 0.28 inches (7 millimeters).

Wherein shunting blood from the left atrium to the coronary sinus through the flow path of the shunt device further includes maintaining a pulmonary to systemic flow ratio between 1.2 and 1.4.

Wherein shunting blood from the left atrium to the coronary sinus through the flow path of the shunt device further includes maintaining a pulmonary to systemic flow ratio around 1.2.

The method further includes increasing a pressure of the blood in the coronary sinus as the blood is shunted through the shunt device from the left atrium to the coronary sinus.

The method further includes dilating the coronary sinus as blood is shunted through the shunt device from the left atrium to the coronary sinus.

Wherein positioning the shunt device in the tissue wall between the left atrium and the coronary sinus of the heart further includes positioning the shunt device in the tissue wall between the left atrium and the coronary sinus of the heart adjacent to a mid-portion of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart adjacent to region P2 and region P3 of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart adjacent to region P2 and region P3 of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 1.18 inches (30 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.98 inches (25 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.79 inches (20 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart 0.79 inches (20 millimeters) from an ostium of the coronary sinus.

The method further includes sterilizing the shunt device.

The above method(s) can be performed on a living animal or on a simulation, such as on a cadaver, cadaver heart, anthropomorphic ghost, simulator (e.g., with body parts, heart, tissue, etc. being simulated).

A method of shunting blood between a left atrium and a right atrium of a heart includes positioning a shunt device in a tissue wall between the left atrium and a coronary sinus of the heart so that a flow path through a central flow tube of the shunt device guides a left-sided flow vortex of blood flow within the left atrium through the central flow tube of the shunt device and is positioned to guide the flow of blood through the flow path of the shunt device to join with a helical flow pattern of blood flow within the coronary sinus. The blood is shunted from the left atrium to the coronary sinus through the flow path of the shunt device. The flow of blood through the shunt device is joined with the helical flow pattern of blood flow within the coronary sinus. The blood from the coronary sinus is moved into the right atrium. The flow of blood from the coronary sinus is joined with a natural flow pattern of blood flow within the right atrium.

The method of the preceding paragraph can optionally include, additionally and/or alternatively, any one or more of the following features, configurations and/or additional components:

Wherein joining the flow of blood from the coronary sinus with the natural flow pattern of blood flow within the right atrium further includes joining the flow of blood from the coronary sinus with a right-sided flow vortex of blood flow within the right atrium.

The method further includes enhancing the right-sided flow vortex of blood flow within the right atrium.

The method further includes reestablishing the right-sided flow vortex of blood flow within the right atrium.

Wherein the blood moving from the coronary sinus into the right atrium includes a natural flow of blood from the coronary sinus and a shunted flow of blood from the left atrium into the coronary sinus through the flow path of the shunt device.

Wherein joining the flow of blood from the coronary sinus with the natural flow pattern of blood flow within the right atrium further includes joining the flow of blood from the coronary sinus with an inflow of blood from an inferior vena cava that flows into the natural flow pattern of blood flow within the right atrium.

Wherein positioning the shunt device in the tissue wall between the left atrium and the coronary sinus of the heart further includes positioning the shunt device at an angle with respect to the tissue wall.

Wherein the angle is between 150 and 90°.

Wherein the angle is between 300 and 75°.

Wherein the angle is between 600 and 65°.

Wherein the central flow tube of the shunt device has a diameter between 0.04 inches (1 millimeters) and 0.47 inches (12 millimeters).

Wherein the central flow tube of the shunt device has a diameter between 0.12 inches (3 millimeters) and 0.39 inches (10 millimeters).

Wherein the central flow tube of the shunt device has a diameter between 0.20 inches (5 millimeters) and 0.32 inches (8 millimeters).

Wherein the central flow tube of the shunt device has a diameter of 0.28 inches (7 millimeters).

Wherein shunting blood from the left atrium to the coronary sinus through the flow path of the shunt device further includes maintaining a pulmonary to systemic flow ratio between 1.2 and 1.4.

Wherein shunting blood from the left atrium to the coronary sinus through the flow path of the shunt device further includes maintaining a pulmonary to systemic flow ratio around 1.2.

The method further includes increasing a pressure of the blood in the coronary sinus as the blood is shunted through the shunt device from the left atrium to the coronary sinus.

The method further includes dilating the coronary sinus as blood is shunted through the shunt device from the left atrium to the coronary sinus.

Wherein positioning the shunt device in the tissue wall between the left atrium and the coronary sinus of the heart further includes positioning the shunt device in the tissue wall between the left atrium and the coronary sinus of the heart adjacent to a mid-portion of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart adjacent to region P2 and region P3 of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 1.18 inches (30 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.98 inches (25 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.79 inches (20 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart 0.79 inches (20 millimeters) from an ostium of the coronary sinus.

The method further includes sterilizing the shunt device.

The above method(s) can be performed on a living animal or on a simulation, such as on a cadaver, cadaver heart, anthropomorphic ghost, simulator (e.g., with body parts, heart, tissue, etc. being simulated).

A method of shunting blood between a left atrium and a right atrium of a heart includes positioning a shunt device in a tissue wall between the left atrium and a coronary sinus of the heart so that a flow path through a central flow tube of the shunt device guides a natural flow pattern of blood flow with the left atrium through the central flow tube of the shunt device. The blood is shunted from the left atrium to the coronary sinus through the flow path of the shunt device. The flow of blood through the shunt device is joined with a natural flow pattern of blood flow within the coronary sinus. The blood is moved from the coronary sinus into the right atrium, and the flow of blood from the coronary sinus is joined with a natural flow pattern of blood flow within the right atrium.

The method of the preceding paragraph can optionally include, additionally and/or alternatively, any one or more of the following features, configurations and/or additional components:

Wherein joining the flow of blood from the coronary sinus with the natural flow pattern of blood flow within the right atrium further includes joining the flow of blood from the coronary sinus with a right-sided flow vortex of blood flow within the right atrium.

The method further includes enhancing the right-sided flow vortex of blood flow within the right atrium.

The method further includes reestablishing the right-sided flow vortex of blood flow within the right atrium.

Wherein the blood moving from the coronary sinus into the right atrium includes a natural flow of blood from the coronary sinus and a shunted flow of blood from the left atrium into the coronary sinus through the flow path of the shunt device.

Wherein joining the flow of blood from the coronary sinus with the natural flow pattern of blood flow within the right atrium further includes joining the flow of blood from the coronary sinus with an inflow of blood from an inferior vena cava that flows into the natural flow pattern of blood flow within the right atrium.

Wherein positioning the shunt device in the tissue wall between the left atrium and the coronary sinus of the heart further includes positioning the shunt device in the tissue wall between the left atrium and the coronary sinus of the heart so that the flow path through the central flow tube of the shunt device guides a left-sided flow vortex of blood flow within the left atrium through the central flow tube of the shunt device.

Wherein positioning the shunt device in the tissue wall between the left atrium and the coronary sinus of the heart further includes positioning the shunt device in the tissue wall between the left atrium and the coronary sinus of the heart so that the flow path through the central flow tube of the shunt device is positioned to guide the flow of blood through the flow path of the shunt device to join with the natural flow pattern of blood flow within the coronary sinus.

Wherein positioning the shunt device in the tissue wall between the left atrium and the coronary sinus of the heart further includes positioning the shunt device in the tissue wall between the left atrium and the coronary sinus of the heart so that the flow path through the central flow tube of the shunt device is positioned to guide the flow of blood through the flow path of the shunt device to join with a helical flow pattern of blood flow within the coronary sinus.

Wherein joining the flow of blood through the shunt device with the natural flow pattern of blood flow within the coronary sinus further includes joining the flow of blood through the shunt device with a helical flow pattern of blood flow within the coronary sinus.

Wherein positioning the shunt device in the tissue wall between the left atrium and the coronary sinus of the heart so that the flow path through the central flow tube of the shunt device guides the natural flow pattern of blood in the left atrium through the central flow tube of the shunt device further includes positioning the shunt device at an angle with respect to the tissue wall.

Wherein the angle is between 150 and 90°.

Wherein the angle is between 300 and 75°.

Wherein the angle is between 600 and 65°.

Wherein the central flow tube of the shunt device has a diameter between 0.04 inches (1 millimeters) and 0.47 inches (12 millimeters).

Wherein the central flow tube of the shunt device has a diameter between 0.12 inches (3 millimeters) and 0.39 inches (10 millimeters).

Wherein the central flow tube of the shunt device has a diameter between 0.20 inches (5 millimeters) and 0.32 inches (8 millimeters).

Wherein the central flow tube of the shunt device has a diameter of 0.28 inches (7 millimeters).

Wherein shunting blood from the left atrium to the coronary sinus through the flow path of the shunt device further includes maintaining a pulmonary to systemic flow ratio between 1.2 and 1.4.

Wherein shunting blood from the left atrium to the coronary sinus through the flow path of the shunt device further includes maintaining a pulmonary to systemic flow ratio around 1.2.

The method further includes increasing a pressure of the blood in the coronary sinus as blood is shunted through the shunt device from the left atrium to the coronary sinus.

The method further includes dilating the coronary sinus as blood is shunted through the shunt device from the left atrium to the coronary sinus.

Wherein positioning the shunt device in the tissue wall between the left atrium and the coronary sinus of the heart further includes positioning the shunt device in the tissue wall between the left atrium and the coronary sinus of the heart adjacent to a mid-portion of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart adjacent to region P2 and region P3 of a posterior leaflet of a mitral valve.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 1.18 inches (30 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.98 inches (25 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart between 0.59 inches (15 millimeters) and 0.79 inches (20 millimeters) from an ostium of the coronary sinus.

Wherein positioning the shunt device in the tissue wall between the left atrium and the coronary sinus of the heart further includes positioning the shunt device in the tissue wall between the left atrium and the coronary sinus of the heart 0.79 inches (20 millimeters) from an ostium of the coronary sinus.

The method further includes sterilizing the shunt device.

The above method(s) can be performed on a living animal or on a simulation, such as on a cadaver, cadaver heart, anthropomorphic ghost, simulator (e.g., with body parts, heart, tissue, etc. being simulated).

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood flow within a coronary sinus.

The shunt device of the preceding paragraph can optionally include, additionally and/or alternatively, any one or more of the following features, configurations and/or additional components:

The shunt device further includes a sensor attached to the shunt device body.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood flow within a left atrium.

The shunt device of the preceding paragraph can optionally include, additionally and/or alternatively, any one or more of the following features, configurations and/or additional components:

The shunt device further includes a sensor attached to the shunt device body.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. When the shunt device is secured to the tissue wall, the central flow tube is angled between 150 and 900 with respect to the tissue wall.

The shunt device of the preceding paragraph can optionally include, additionally and/or alternatively, any one or more of the following features, configurations and/or additional components:

The shunt device further includes a sensor attached to the shunt device body.

Wherein when the shunt device is secured to the tissue wall, the central flow tube is angled between 300 and 750 with respect to the tissue wall.

Wherein when the shunt device is secured to the tissue wall, the central flow tube is angled between 600 and 650 with respect to the tissue wall.

A shunt device includes a shunt device body formed of a plurality of struts. The shunt device body includes a central flow tube, a flow path extending through the central flow tube, and a plurality of arms extending outward from the flow tube and configured to secure the shunt device to a tissue wall. The flow path has a diameter between 0.12 inches (3 millimeters) and 0.39 inches (10 millimeters). When the shunt device is secured to the tissue wall, the central flow tube of the shunt device is positioned at an angle with respect to the tissue wall so that the central flow tube of the shunt device is configured to guide a flow of blood through the central flow tube of the shunt device to join a natural flow pattern of blood in a coronary sinus.

The shunt device of the preceding paragraph can optionally include, additionally and/or alternatively, any one or more of the following features, configurations and/or additional components:

The shunt device further includes a sensor attached to the shunt device body.

Wherein the flow path has a diameter between 0.20 inches (5 millimeters) and 0.32 inches (8 millimeters).

Wherein the flow path has a diameter of 0.28 inches (7 millimeters).

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A method of selecting a shunt device for implantation in a heart, the method comprising:
obtaining a first MRI of the heart;
generating a simulation of flow patterns of blood flow in the heart;
simulating blood flow in the heart when various shunt devices are implanted in the heart;
selecting the shunt device that complements the flow patterns of blood flow in the heart; and
implanting the shunt device in the heart.

2. The method of claim 1, wherein obtaining the first MRI of the heart further comprises:
obtaining a 4D MRI of the heart to visualize the flow patterns of blood flow in the heart.

3. The method of claim 1, wherein obtaining the first MRI of the heart further comprises:
obtaining a 4D MRI of the heart to visualize the flow patterns of blood flow in a right atrium, a left atrium, and/or a coronary sinus of the heart.

4. The method of claim 1, wherein generating the simulation of the flow patterns of blood flow in the heart further comprises:
generating a simulation of the flow patterns of blood flow in a right atrium, a left atrium, and/or a coronary sinus of the heart.

5. The method of claim 1, wherein simulating blood flow in the heart when the various shunt devices are implanted in the heart further comprises:
simulating blood flow in the heart when the shunt devices having varying cross-sectional areas of a flow path of the shunt device, varying angles of central flow tubes of the shunt devices with respect to a tissue wall in which the shunt devices are implanted, and/or varying placement of the shunt devices along a coronary sinus are implanted in the heart.

6. The method of claim 1, wherein selecting the shunt device that complements the flow patterns of blood flow in the heart further comprises:
selecting a design of the shunt device that complements the flow patterns of blood flow in the heart.

7. The method of claim 6, wherein selecting the design of the shunt device that complements the flow patterns of blood flow in the heart further comprises:
selecting a cross-sectional area of a flow path of the shunt device that complements the flow patterns of blood flow in the heart.

8. The method of claim 6, wherein selecting the design of the shunt device that complements the flow patterns of blood flow in the heart further comprises:
selecting an angle of a central flow tube of the shunt device with respect to the tissue wall in which the shunt device is implant that complements the flow patterns of blood flow in the heart.

9. The method of claim 6, wherein selecting the shunt device that complements the flow patterns of blood flow in the heart further comprises:
selecting the design of the shunt device that complements a right-sided flow vortex in a right atrium of the heart.

10. The method of claim 1, wherein selecting the shunt device that complements the flow patterns of blood flow in the heart further comprises:
selecting a placement of the shunt device along a coronary sinus that complements the flow patterns of blood flow in the heart.

11. The method of claim 10, wherein selecting the placement of the shunt device along the coronary sinus that complements the flow patterns of blood flow in the heart further comprises:
selecting a placement of the shunt device along the coronary sinus that complements a right-sided flow vortex in a right atrium of the heart.

12. The method of claim 1, wherein selecting the shunt device that complements the flow patterns of blood flow in the heart further comprises:
selecting the shunt device that complements a right-sided flow vortex in a right atrium of the heart, a left-sided flow vortex in a left atrium of the heart, and/or a helical flow pattern of blood flow in a coronary sinus of the heart.

13. The method of claim 1, wherein selecting the shunt device that complements the flow patterns of blood flow in the heart further comprises:
selecting a design of the shunt device and a placement of the shunt device along a coronary sinus that enhances a right-sided flow vortex of blood flow in a right atrium of the heart.

14. The method of claim 1, wherein selecting the shunt device that complements the flow patterns of blood flow in the heart further comprises:
selecting a design of the shunt device and a placement of the shunt device along a coronary sinus that reestablishes a right-sided flow vortex of blood flow in a right atrium of the heart.

15. The method of claim 1, and further comprising:
obtaining a second MRI of the heart.

16. The method of claim 15, wherein obtaining the second MRI of the heart further comprises:
obtaining a 4D MRI of the heart to visualize the flow patterns of blood flow in the heart.

17. The method of claim 15, wherein obtaining the 4D MRI of the heart to visualize the flow patterns of blood flow in the heart further comprises:
obtaining the 4D MRI of the heart to visualize the flow patterns of blood flow in a right atrium, a left atrium, and/or a coronary sinus of the heart.

18. The method of claim 15, wherein obtaining the first MRI of the heart further comprises:
measuring a volume of one or more chambers of the heart;
measuring a size of one or more chamber of the heart;
measuring a geometry of one or more chambers of the heart;
measuring a compliance of one or more chambers of the heart;
measuring a blood pressure in one or more chambers of the heart;
tracking a movement of one or more chambers of the heart; and/or
tracking a movement of a tricuspid valve of the heart; and
wherein obtaining the second MRI of the heart further comprises:
measuring a volume of one or more chambers of the heart;
measuring a size of one or more chamber of the heart;
measuring a geometry of one or more chambers of the heart;
measuring a compliance of one or more chambers of the heart;
measuring a blood pressure in one or more chambers of the heart;
tracking a movement of one or more chambers of the heart; and/or
tracking a movement of a tricuspid valve of the heart.

19. The method of claim 18, and further comprising:
comparing one or more of the volume, the size, the geometry, the compliance, the blood pressure, the movement of one or more chambers, and/or the movement of the tricuspid valve from the first MRI with one or more of the volume, the size, the geometry, the compliance, the blood pressure, the movement of one or more chambers, and/or the movement of the tricuspid valve from the second MRI to analyze an overall health of the heart.

20. The method of claim 1, and further comprising:
adjusting the shunt device.
